(19) United States
(12) Patent Application Publication
(10) Pub. No.: US 2020/0362057 A1
(43) Pub. Date: Nov. 19, 2020

(54) COMPOSITIONS AND METHODS FOR THE TREATMENT OF MAJOR DEPRESSIVE DISORDER

(71) Applicant: Snake River Bioscience, Inc., Wilson, WY (US)

(72) Inventor: Erich Gulbins, Essen (DE)

(21) Appl. No.: 16/860,713

(22) Filed: Apr. 28, 2020

Related U.S. Application Data
(60) Provisional application No. 62/848,816, filed on May 16, 2019.

(51) Int. Cl. C07K 16/44; A61P 25/24

(52) U.S. Cl. CPC C07K 16/44; A61K 45/06; A61P 25/24

(57) ABSTRACT

Provided herein are methods of treating major depressive disorder (MDD) in a subject in need thereof, the method including administering to the subject an effective amount of an active agent that reduces an amount of ceramide in a peripheral compartment of the subject. Also provided are methods of reducing ceramide levels in neuronal tissue of a subject suffering from MDD, the method including peripherally administering to the subject an effective amount of an active agent that modulates ceramide; as well as methods of identifying a candidate agent to treat MDD.

### CROSS-REFERENCE TO RELATED APPLICATIONS

This application claims priority to U.S. Provisional Application Ser. No. 62/848,816, filed May 16, 2019, the entire contents of which are incorporated by reference.

### TECHNICAL FIELD

This disclosure is directed to compositions and methods for the treatment of major depressive disorder.

### BACKGROUND

Major depressive disorder (MDD) is a severe and chronic disease with a lifetime prevalence of more than 10%. This disease is also often a life-threatening illness with suicide as cause of death for an estimated 10% of patients with severe MDD. Besides a depressed mood, the disease is characterized by a loss of interest, anhedonia, fear, feelings of worthlessness, weight loss, insomnia, and concentration deficits. MDD is also associated with many somatic symptoms, such as increased incidence of cardiovascular disease and osteoporosis, adrenocortical activation, increased oxidative stress, and increased plasma concentrations of proinflammatory cytokines and phospholipase A₂, as well as dyslipoproteinemia.

Current treatments of MDD include the use of antidepressants, sleep deprivation, or electroconvulsive therapy. The previously held monoamine hypothesis for the actions of antidepressants was recently revised because the antidepressant effect is not clearly correlated with the monoaminergic effect of the drugs; in fact, the antidepressant tianeptine is even a serotonin reuptake enhancer. Furthermore, the direct and rapid effect of antidepressants on the synaptic uptake of monoamines is in contrast to the delayed antidepressant effects of these drugs, which usually exhibit a clinical effect only after 2 to 4 weeks. Thus, it has been suggested that a defect in or a reduction of neurogenesis in the hippocampus is a central element of the disease. This notion is supported by the finding that chronic stress and depression, in both rodents and humans, result in hippocampal atrophy, which is reversed by 2 to 3 weeks of treatment with antidepressants, a finding consistent with the delayed action of antidepressants. In stress-induced models of MDD, antidepressants have been shown to increase cell proliferation and neurogenesis in primary neural cultures in vitro and in the hippocampus of adult rodents in vivo, and to improve behavior. Inhibition or ablation of neurogenesis, e.g., by selective irradiation of the hippocampus, on the other hand, does not result in MDD, questioning the role of neurogenesis in the pathogenesis of major depression. Furthermore, electroconvulsive therapy, an alternative treatment for MDD, and several recently developed medications such as ketamine have a rapid therapeutic effect, which is also inconsistent with an extended length of neurogenesis and maturation as a requirement for antidepressive therapy. Thus, the pathogenesis of major depression is still unknown. A need exists for new compositions and methods for the treatment of MDD.

### SUMMARY

The present disclosure elucidates the role of ceramide in the pathogenesis of major depressive disorder (MDD). Advantageously, the present investigators found that reducing a concentration of ceramide in the periphery, i.e. outside the central nervous system, of a subject is effective to treat or abrogate MDD and/or one or more symptoms thereof.

In one aspect, a method of treating MDD or a composition for use in treating MDD in a subject in need thereof is provided, comprising administering to the subject an effective amount of an active agent that reduces an amount of ceramide in a peripheral compartment of the subject.

In another aspect, a method of reducing ceramide levels in neuronal tissue of a subject suffering from MDD is provided, the method comprising peripherally administering to the subject an effective amount of an active agent that modulates ceramide or ceramide levels.

In another aspect, a method for identifying a candidate agent to treat MDD is provided, the method comprising: contacting ceramide with a test compound in a first sample; contacting ceramide with a control in a second sample; and measuring a concentration of functional ceramide in the first and second samples, wherein a reduction in the concentration of functional ceramide in the first sample compared to the second sample indicates that test compound is a candidate agent for treating MDD.

In another aspect, a method for identifying a candidate agent to treat MDD is provided, the method comprising: contacting an intact cell capable of synthesizing/releasing ceramide with a test compound in a first sample; contacting an intact cell capable of synthesizing ceramide with a positive control or a negative control in a second sample; and measuring a concentration of ceramide in the first and second samples, wherein a reduction in the concentration of ceramide in the first sample compared to the second sample indicates that the test compound is a candidate agent for treating MDD.

These and other objects, features, embodiments, and advantages will become apparent to those of ordinary skill in the art from a reading of the following detailed description and the appended claims.

### BRIEF DESCRIPTION OF THE DRAWINGS

FIG. 1a shows ceramide concentration in wildtype mouse plasma as determined by mass spectrometry (left panel) and kinase assay (right panel). Results indicate stress induces a marked increase in ceramide concentration in the blood plasma of wildtype mice.

FIG. 1b shows ceramide concentration in human blood plasma of patients with MDD compared to healthy individuals, as determined by mass spectrometry (left panel) and kinase assay (right panel). Results indicate an increase in ceramide concentrations in the plasma of patients with MDD compared to healthy individuals.

FIG. 1c shows ceramide concentration in wildtype and Nsm^{-/-} mouse exosomes, as determined by mass spectrometry (left panel) and kinase assay (right panel). Results indicate glucocorticosterone-mediated or chronic unpredictable environmental stress (CUS) induces a marked increase in ceramide concentrations in exosomes in the blood plasma of mice.

FIG. 1d shows ceramide concentration in exosomes obtained from plasma of human patients with MDD compared to healthy individuals, as determined by mass spectrometry (left panel) and kinase assay (right panel). Results indicate ceramide concentration in exosomes of MDD patients is about three times higher than ceramide concentration in exosomes of healthy control subjects.

FIG. 2a shows behavior of stressed mice (exposed to glucocorticosterone or environmental stress treated with anti-ceramide IgM antibodies clone S58-9, recombinant neutral ceramidase, or IgM control antibody. Observed behavior included time outside box (top left panel), latency to feed (top right panel), time in center (middle left panel), splash test (middle right panel), forced swim test (bottom left panel), and coat test (bottom right panel). Results indicate that neutralizing or consuming ceramide abrogates behavioral signs of MDD in stressed mice.

FIG. 2b shows BrdU-positive cells as a marker of neurogenesis in stressed mice exposed to glucocorticosterone or CUS treated with anti-ceramide IgM antibodies clone S58-9, recombinant neutral ceramidase, or IgM control antibody. Results indicate that neutralizing or consuming ceramide normalizes neurogenesis in stressed mice.

FIG. 3a shows behavior of un-stressed mice injected with plasma from stressed mice (exposed to glucocorticosterone or CUS), wherein plasma was incubated with anti-ceramide IgM antibodies clone S58-9, recombinant neutral ceramidase, IgM control antibody, or untreated. Observed behavior included time outside box (top left panel), latency to feed (top right panel), time in center (middle left panel), splash test (middle right panel), forced swim test (bottom left panel), and coat test (bottom right panel). Results indicate injection of blood plasma from stressed mice into non-stressed, healthy mice transferred the symptoms of MDD and in vitro incubation of plasma from stressed mice with anti-ceramide antibodies clone S58-9 or ceramidase prior to re-injection into non-stressed mice prevented development of behavioral signs of MDD.

FIG. 3b shows BrdU-positive cells as a marker of neurogenesis in un-stressed mice injected with plasma from stressed mice, wherein the plasma was incubated with anti-ceramide IgM antibodies clone S58-9, recombinant neutral ceramidase, IgM control antibody, or untreated. Results indicate injection of blood plasma from stressed mice into non-stressed, healthy mice transferred the symptoms of MDD and in vitro incubation of plasma from stressed mice with anti-ceramide antibodies clone S58-9 or ceramidase prior to re-injection into non-stressed mice normalized neurogenesis.

FIG. 3c shows behavioral effects of injection of isolated exosomes from stressed mice, optionally incubated with anti-ceramide IgM antibodies clone S58-9, recombinant neutral ceramidase, IgM control antibody, or untreated, and re-injection into healthy control mice. Observed behavior included time outside box (top left panel), latency to feed (top right panel), time in center (middle left panel), splash test (middle right panel), forced swim test (bottom left panel), and coat test (bottom right panel). Results indicate exosomes purified from stressed mice induced depressive behavior in healthy mice, as evidenced by behavioral markers of MDD.

FIG. 3d shows BrdU-positive cells as a marker of neurogenesis after isolation of exosomes from stressed mice, optionally incubated with anti-ceramide IgM antibodies clone S58-9, recombinant neutral ceramidase, IgM control antibody, or untreated, and re-injection into healthy control mice. Results indicate exosomes purified from stressed mice reduced neuronal proliferation in healthy mice, as evidenced by BrdU-positive cells.

FIG. 4a shows behavioral effects of stress (exposed to glucocorticosterone or CUS) on mice deficient in neutral sphingomyelinase 2 or wildtype mice. Observed behavior included time outside box (top left panel), latency to feed (top middle panel), time in center (top right panel), splash test (bottom left panel), forced swim test (bottom middle panel), and coat test (bottom right panel). Results indicate stress did not induce changes in behavior in mice deficient in neutral sphingomyelinase 2, whereas stress exerted strong effects in wildtype littermates.

FIG. 4b shows neurogenesis in mice deficient in neutral sphingomyelinase 2 or wildtype mice exposed to glucocorticosterone or CUS. Results indicate stress only slightly reduced neurogenesis in mice deficient in neutral sphingomyelinase 2, whereas stress exerted strong effects in wildtype littermates.

FIG. 4c shows behavioral effects of injection into wildtype mice of plasma or exosomes isolated from stressed mice deficient in neutral sphingomyelinase 2 (exposed to glucocorticosterone or environmental stress). Behavioral studies included time outside box (top left panel), latency to feed (top middle panel), time in center (top right panel), splash test (bottom left panel), forced swim test (bottom middle panel), and coat test (bottom right panel). Results indicate injecting plasma or exosomes isolated from stressed mice deficient in neutral sphingomyelinase 2 into untreated wildtype mice did not induce any signs of major depression, whereas injecting exosomes from stressed wildtype littermates induced behavioral signs of MDD within 24 h.

FIG. 4d shows BrdU-positive cells as a marker of neurogenesis in wildtype mice injected with plasma or exosomes isolated from stressed mice deficient in neutral sphingomyelinase 2. Results indicate injecting plasma or exosomes isolated from stressed mice deficient in neutral sphingomyelinase 2 into untreated wildtype mice did not induce any signs of major depression, whereas injecting exosomes from stressed wildtype littermates reduced neuronal proliferation within 24 h.

FIG. 4e shows behavioral and neural effects of increasing ceramide concentration in untreated mice. Plasma or exosomes from untreated mice was loaded with C₁₆-ceramide, injected into non-stressed mice, and behavioral or neural changes (as measured by time outside box (top left panel), latency to feed (top middle panel), splash test (top right panel), time in center (bottom left panel), forced swim test (bottom middle panel), and neurogenesis (bottom right panel)) were assessed. Results indicate that loading the blood plasma or exosomes with C₁₆-ceramide is sufficient to induce behavioral signs of MDD within 24 h.

FIG. 5a shows behavioral effects of injection of exosomes isolated from blood plasma of depressed human patients into healthy mice, wherein human exosomes were optionally incubated with anti-ceramide antibodies clone S58-9, ceramidase, or IgM control. Observed behavior included time outside box (top left panel), latency to feed (top right panel), time in center (middle left panel), splash test (middle right panel), swim test (bottom left panel), and coat test (bottom right panel). Results indicate that exosomes from depressed patients, but not healthy individuals, induced depressive behavior in healthy mice as early as 24 h after injection. Further, pre-incubation with anti-ceramide antibodies clone S58-9 or ceramidase prevented the effects on mice, while control IgM exerted no effect.

FIG. 5b shows neurogenic effects of injection of exosomes isolated from blood plasma of depressed human patients into healthy mice, wherein human exosomes were optionally incubated with anti-ceramide antibodies clone S58-9, ceramidase, or IgM control. Results as measured by BrdU positive cells indicate that exosomes from depressed patients, but not healthy individuals, reduced neurogenesis in healthy mice as early as 24 h after injection. Further, pre-incubation with anti-ceramide antibodies clone S58-9 or ceramidase prevented the effects on mice, while control IgM exerted no effect.

FIG. 6 shows behavior of stressed (exposed to glucocorticosterone or environmental stress) or unstressed mice treated with monoclonal mouse anti-ceramide IgG, monoclonal mouse anti-ceramide IgM clone S58-9, or IgG control antibody. Observed behavior included time outside box (top left panel), latency to feed (top right panel), time in center (middle left panel), splash test (middle right panel), forced swim test (bottom left panel), and coat test (bottom right panel). Results indicate neutralizing or consuming ceramide abrogates behavioral signs of major depression in stressed mice. Injection of control IgG did not alter depressive behavior.

FIG. 7 shows behavior of un-stressed mice injected with plasma from stressed mice (exposed to glucocorticosterone or CUS), wherein the plasma was incubated in vitro with monoclonal mouse anti-ceramide IgG, monoclonal mouse anti-ceramide IgM clone MID 15B4, IgM control antibody, IgG control antibody, or untreated. Observed behavior included time outside box (top left panel), latency to feed (top right panel), time in center (middle left panel), splash test (middle right panel), forced swim test (bottom left panel), and coat test (bottom right panel). Results indicate injection of blood plasma from stressed mice into non-stressed, healthy mice transferred the symptoms of MDD and in vitro incubation of plasma from stressed mice with anti-ceramide antibodies prior to re-injection into non-stressed mice prevented induction of behavioral signs of MDD.

### DETAILED DISCLOSURE

Provided herein are new methods for the prevention or treatment, or compositions for the prevention or treatment of disorders such as major depressive disorder (MDD) based on a reduction of peripheral ceramide levels that were surprisingly found to reduce measures of MDD.

“Peripheral” is defined herein as within the body of a subject not within the central nervous system. Illustrative examples of peripheral compartments include but are not limited to blood, immune organs, endothelial cells, liver, spleen, etc. The methods as provided herein are appreciated to utilize molecules that either directly reduce peripheral (e.g., blood) ceramide levels and/or alter ceramide synthesis and/or degradation so that peripheral levels of ceramide are thereby reduced relative to the levels in that compartment prior to treatment.

Acid sphingomyelinase (abbreviated ASM for the human protein, Asm for the mouse protein; EC 3.1.4.12, sphingomyelin phosphodiesterase, optimum pH 5.0; gene symbol, Smpd1) is a glycoprotein that functions as a lysosomal hydrolase, catalyzing the degradation of sphingomyelin to phosphorylcholine and ceramide. Genetic deficiency of Asm abrogates the effects of some antidepressants on neurogenesis and behavior, whereas mice overexpressing Asm exhibit constitutive changes similar to those associated with mild MDD. Studies showed that amitriptyline and fluoxetine induce autophagy in hippocampal neurons via a slow accumulation of sphingomyelin in lysosomes and Golgi bodies and of ceramide in the endoplasmic reticulum (ER). Gulbins, et al., Molecular Psychiatry (2018) 23:2324-2346. ER ceramide stimulates phosphatase 2A (PP2A), Ulk, Beclin, PI3K/Vps34, p62 and Lc3B and thereby autophagy and the formation of autophagolysosomes. Direct inhibition of sphingomyelin synthases with D609 results in rapid accumulation of ceramide in the ER, activation of autophagy and rapid reversal of stress-induced MDD. Inhibition of Beclin blocked the antidepressive effects of amitriptyline and D609 and induces cellular and behavioral changes typical of MDD. Although these studies identified molecular mechanisms by which certain antidepressants act, they do not identify molecular mechanisms that cause major depression.

While not desiring to be bound by theory, the inventors propose herein a novel approach to treating MDD. At present, MDD is understood as a central nervous system disease with some consequences in the periphery (i.e., any area other than the central nervous system). This accounts for the historical understanding in the art that ceramide levels were, at best, merely a biomarker of antidepressant activity. In contrast, this disclosure is the first to identify peripheral ceramide as a cause for MDD and to advance the solution that compositions and methods that regulate peripheral ceramide levels, and optionally selectively regulate ceramide levels, can be used to treat or prevent MDD or the symptoms thereof in subjects who are suffering from or are at risk for developing MDD.

A basis for the methods as provided herein, without being limited to one particular theory, is the new understanding that MDD is a peripheral disease, optionally in response to stress, with manifestations in the brain, cardiovascular system, or skeletomuscular systems. Stress induces an increase of ceramide within the blood. Ceramide in the blood is contained in exosomes or other vesicles that are able to penetrate the blood brain barrier (BBB) and reach the hippocampus to alter neuronal functions and thereby induce MDD. This new concept also allows for novel treatments of MDD as provided herein; i.e., instead of targeting molecules in the brain, this disclosure prevents the formation of ceramide in the periphery, neutralizes ceramide in the blood plasma, or consumes ceramide in the blood plasma. According to this disclosure, as ceramide released in the periphery induces functional changes in neurons in the brain, this disclosure indicates that neutralization/degradation of ceramide in the blood restores the function of neurons and acts rapidly to reverse major depression.

As such, processes as provided herein include administering to a subject in need a modulator of peripheral ceramide levels in an amount effective to reduce the amount of peripheral ceramide in the subject. Optionally, peripheral ceramide levels are or include the level of circulating ceramide in the blood compartment of the subject. By reducing the levels of peripheral ceramide, the subject is treated for MDD, the subject's risk of developing MDD is reduced, or the subject is prevented from developing MDD, or one or more symptoms of MDD.

As used herein, a “subject” is defined as an organism (such as a human, non-human primate, equine, bovine, murine, or other mammal), or a cell.

A “subject in need” is a subject that exhibits one or more symptoms of MDD (optionally chronically), has been clinically diagnosed as having MDD, or is at risk of developing MDD. Optionally, a subject in need has an elevated level of ceramide in the peripheral blood relative to an average amount as found in a similarly unstressed individual or population of individuals. MDD symptoms may be listed in scores such as the Hamilton Depression score and include: Depressed mood, gloomy attitude, pessimism about the future, feeling of sadness, tendency to weep, feelings of guilt, suicide, initial insomnia, insomnia during the night, delayed insomnia, absence from work, no interests, Retardation (slowness of thought, speech, and activity; apathy; stupor), agitation (restlessness associated with anxiety), pyschiatric anxiety, somatic anxiety (gastrointestinal, indigestion, cardiovascular, palpitations, headaches, respiratory, genitourinary, etc), gastrointestinal somatic symptoms (loss of appetite, heavy feeling in abdomen, constipation), general somatic symptoms (heaviness in limbs, back, or head; diffuse backache; loss of energy and fatigability), genital symptoms (loss of libido, menstrual disturbances), hypochondriasis, weight loss, lack of insight (patient's understanding). Illustrative symptoms of MDD in a subject in need include, but are not limited to: feelings of sadness, tearfulness, emptiness, or hopelessness; angry outbursts, irritability or frustration, often over small matters; loss of interest or pleasure in most or all normal activities illustratively sex, hobbies, or sports; sleep disturbances, including insomnia or excessive sleeping; tiredness or lack of energy; reduced appetite or weight loss, cravings for food or weight gain; anxiety, agitation or restlessness; slowed thinking, speaking or body movements; feelings of worthlessness or guilt or self-blame; trouble thinking, concentrating, making decisions or remembering things; frequent recurrent thoughts of death, suicidal thoughts, suicide attempts; unexplained physical problems such as back pain or headaches; or any combination thereof or parts thereof. In some aspects, a subject in need is one who has been clinically diagnosed as suffering from MDD. In some aspects, a subject in need is one who has historically been diagnosed with MDD, but is not currently suffering from the condition. In some aspects, a subject in need is one who is at risk for developing MDD.

Major depressive disorder (MDD), or major depression, as used herein, includes endogenous MDD, which manifests without an external stressful event or trigger, as well as reactive or exogenous MDD, wherein an external stressful event or trigger is identifiable.

Methods as provided herein optionally include administering to the subject an effective amount of one or more active agents that will reduce an amount of ceramide in a cell, the blood, or other peripheral compartment of a subject. Illustratively, an active agent is an antibody, nucleic acid, peptide, protein, aptamer, small molecule (e.g. <1000 Da), or other such compound. Optionally, an active agent has as its sole biological activity promoting a reduction in the presence of or functionality of ceramide in one or more peripheral compartments where ceramide can be found.

In some aspects, an active agent is an antibody, optionally a humanized antibody. Illustrative examples of an antibody active agent can be found in U.S. Pat. Nos. 9,592,238 and 10,450,385. Illustrative examples of an antibody include: a. a human antibody; b. a humanized antibody; c. a chimeric antibody; d. a monoclonal antibody; e. a polyclonal antibody; f. a recombinant antibody; g. an antigen-binding antibody fragment; h. a single chain antibody; i. a diabody; j. a triabody; k. a tetrabody; l. a Fab fragment; m. a F(ab′)₂ fragment; n. a domain antibody; o. an IgD antibody; p. an IgE antibody; q. an IgM antibody; r. an IgG1 antibody; s. an IgG2 antibody; t. an IgG3 antibody; u. an IgG4 antibody; v. an IgG4 antibody having at least one mutation in a hinge region that alleviates a tendency to form intra-H chain disulfide bond; or w. a single-chain variable fragment (scFv).

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. Optionally, certain amino acids in the framework and constant domains of the heavy and/or light chains of the non-human species antibody may be mutated to produce the humanized antibody. Optionally, the constant domain(s) from a human antibody are fused to the variable domain(s) of a non-human species. Optionally, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “CDR” refers to the “complementarity determining region” of an immunoglobulin (antibody) molecule. CDRs are part of the variable domain in an antibody where the antibody binds to its specific antigen. CDRs are crucial to the diversity of antigen specificities generated by lymphocytes. There are three CDR per variable domain (i.e., CDR1, CDR2 and CDR3 in the variable domain of the light chain and CDR1, CDR2 and CDR3 in the variable domain of the heavy chain) for a total of 12 CDRs in an IgG molecule and 60 CDRs in an IgM molecule.

In some aspects, the anti-ceramide antibody or fragment comprises: a heavy chain variable region CDR1 of 10 amino acids comprising a Gly in the 1st position from the N-terminal, a Tyr or Phe in the 2nd position from the N-terminal, a Phe or Leu in the 4th position from the N-terminal, and a Thr or His in the 6th position from the N-terminal and a His or Asn in the 10th position from the N-terminal; a heavy chain variable region CDR2 of 16-17 amino acids comprising a Asn or Ile in the 2nd position from the N-terminal, a Phe or Ser in the 4th position from the N-terminal, a Thr in the 9th position from the C-terminal, a Tyr in the 7th position from the C-terminal, an Asn in the 6th position from the C-terminal, a Lys or Ala in the 2nd and 4th positions from the C-terminal; a heavy chain variable region CDR3 of 7 to 11 amino acids comprising a Tyr or Thr at the 4th position from the N-terminal; a light chain variable region CDR1 of 10-16 amino acids comprising an Ala or Ser in the 2nd position from the N-terminal, a Ser in the 3rd position from the N-terminal, a Ser or Asp in the 5th position from the N-terminal, and a Tyr, Ser or Phe in the 3rd position from the C-terminal; a light chain variable region CDR2 of 7 amino acids comprising a Ser or Asn in the 3rd position from the N-terminal, a Lys or Ser in the 5th position from the N-terminal and a Ser or Asp in the 7th position from the N-terminal; and a light chain variable region CDR3 of 9 amino acids comprising a Gln, Leu or Trp in the 1st position from the N-terminal, a Gln in the 2nd position from the N-terminal, a Pro in the 7th position from the N-terminal and a Thr in the 9th position from the N-terminal.

In some aspects, the heavy chain variable region CDR1 of the anti-ceramide antibody, or antigen-binding fragment thereof, comprises the sequence GYTFTDHTIH (SEQ ID NO: 1), said heavy chain variable region CDR2 comprises the sequence YNYPRDGSTKYNEKFKG (SEQ ID NO: 2), said heavy chain variable region CDR3 comprises the sequence GFITTVVPSAY (SEQ ID NO: 3), said light chain variable region CDR1 comprises the sequence RASKSISKYLA (SEQ ID NO: 4), said light chain variable region CDR2 comprises the sequence SGSTLQS (SEQ ID NO: 5), and said light chain variable region CDR3 comprises the sequence QQHNEYPWT (SEQ ID NO: 6).

In other aspects, the heavy chain variable region CDR1 of the anti-ceramide antibody, or antigen-binding fragment thereof, comprises the sequence GYAFSSYWIVIN (SEQ ID NO: 7), said heavy chain variable region CDR2 comprises the sequence QIYPGDGDTNYNGKFKG (SEQ ID NO: 8), said heavy chain variable region CDR3 comprises the sequence RCYYGLYFDV (SEQ ID NO: 9), said light chain variable region CDR1 comprises the sequence KASQDINRYLS (SEQ ID NO: 10), said light chain variable region CDR2 comprises the sequence RANRLVD (SEQ ID NO: 11), and said light chain variable region CDR3 comprises the sequence LQYDEFPYT (SEQ ID NO: 12).

In other aspects, the heavy chain variable region CDR1 of the anti-ceramide antibody, or antigen-binding fragment thereof, comprises the sequence GYTFTSYWMH (SEQ ID NO: 13), said heavy chain variable region CDR2 comprises the sequence YINPSSGYTKYNQFKD (SEQ ID NO: 14), said heavy chain variable region CDR3 comprises the sequence GGYYGFAY (SEQ ID NO: 15), said light chain variable region CDR1 comprises the sequence SASSSVSYMY(SEQ ID NO: 16), said light chain variable region CDR2 comprises the sequence LTSNLAS (SEQ ID NO: 17), and said light chain variable region CDR3 comprises the sequence QQWSSNPLT (SEQ ID NO: 18).

In other aspects, the heavy chain variable region CDR1 of the anti-ceramide antibody, or antigen-binding fragment thereof, comprises the sequence GFSLTGYGVH (SEQ ID NO: 19), said heavy chain variable region CDR2 comprises the sequence VIWSGGSTDYNAAFIS (SEQ ID NO: 20), said heavy chain variable region CDR3 comprises the sequence NYGYDYAMDY (SEQ ID NO: 21), said light chain variable region CDR1 comprises the sequence RASQSIGTSIH (SEQ ID NO: 22), said light chain variable region CDR2 comprises the sequence YASESIS (SEQ ID NO: 23), and said light chain variable region CDR3 comprises the sequence QQSNSWPFT (SEQ ID NO: 24).

In other aspects, the heavy chain variable region CDR1 of the anti-ceramide antibody, or antigen-binding fragment thereof, comprises the sequence GYTFTNYWMH (SEQ ID NO: 25), said heavy chain variable region CDR2 comprises the sequence AIYPGDSDTSYNQKFKG (SEQ ID NO: 26), said heavy chain variable region CDR3 comprises the sequence GLYYGYD (SEQ ID NO: 27), said light chain variable region CDR1 comprises the sequence KSSQSLIDSDGKTFLN (SEQ ID NO: 28), said light chain variable region CDR2 comprises the sequence LVSKLDS (SEQ ID NO: 29), and said light chain variable region CDR3 comprises the sequence WQGTHFPYT (SEQ ID NO: 30).

In particular aspects, the anti-ceramide antibody is selected from the group consisting of monoclonal antibody, chimeric antibody, humanized antibody, human antibody, recombinant antibody, and single-chain variable fragment (scFv).

In some aspects, the anti-ceramide agent is an anti-ceramide single-chain variable fragment (scFv). The scFv comprises: a heavy chain variable region CDR1 of 10 amino acids comprising a Gly in the 1st position from the N-terminal, a Tyr or Phe in the 2nd position from the N-terminal, a Phe or Leu in the 4th position from the N-terminal, and a Thr or His in the 6th position from the N-terminal and a His or Asn in the 10th position from the N-terminal; a heavy chain variable region CDR2 of 16-17 amino acids comprising an Asn or Ile in the 2nd position from the N-terminal, a Phe or Ser in the 4th position from the N-terminal, a Thr in the 9th position from the C-terminal, a Tyr in the 7th position from the C-terminal, an Asn in the 6th position from the C-terminal, a Lys or Ala in the 2nd and 4th positions from the C-terminal; a heavy chain variable region CDR3 of 7 to 11 amino acids comprising a Tyr or Thr at the 4th position from the N-terminal; a light chain variable region CDR1 of 10-16 amino acids comprising an Ala or Ser in the 2nd position from the N-terminal, a Ser in the 3rd position from the N-terminal, a Ser or Asp in the 5th position from the N-terminal, and a Tyr, Ser or Phe in the 3rd position from the C-terminal; a light chain variable region CDR2 of 7 amino acids comprising a Ser or Asn in the 3rd position from the N-terminal, a Lys or Ser in the 5th position from the N-terminal and a Ser or Asp in the 7th position from the N-terminal; and a light chain variable region CDR3 of 9 amino acids comprising a Gln, Leu or Trp in the 1st position from the N-terminal, a Gln in the 2nd position from the N-terminal, a Pro in the 7th position from the N-terminal and a Thr in the 9th position from the N-terminal.

In other aspects, an active agent as used in the methods as provided herein are or include a ceramide binding protein or peptide, fragment thereof, derivative thereof, or variant thereof. Illustrative examples of ceramide binding proteins or peptides could be fragments derived from kinase substrate of Ras (Ksr), Raf-kinase, protein-kinase C (PKC), VDAC, inhibitor 2 of PP2a or CERT, which are proteins that bind ceramide and could be used, for instance in a fusion protein or a recombinant protein, as a domain to neutralize ceramide. Illustrative examples of other ceramide binding proteins include, but are not limited to those identified by Bidlingmaier, et al., Proteome-wide Identification of Novel Ceramide-binding Proteins by Yeast Surface cDNA Display and Deep Sequencing, Mol. Cell. Proteomics 15(4): 1232-45 (2016), or fragments thereof that are capable of binding to ceramide. Optionally, a ceramide binding protein or peptide is or includes a ceramide-binding domain, fragment thereof, derivative thereof, or variant thereof. Illustrative examples of ceramide binding domains include but are not limited to the EF-hand calcium-binding motif, the heat shock chaperonin-binding motif STI1, the SCP2 sterol-binding domain, and the tetratricopeptide repeat region motif. A ceramide binding protein or peptide is optionally capable of neutralizing ceramide either by reducing the amount of ceramide or the functional amount of ceramide in the blood, illustratively by targeting the ceramide for clearance or actively degrading the ceramide. Ceramide neutralization is understood as either preventing transport of ceramide across the blood-brain barrier, removing ceramide from the blood such as by degradation or other mechanism, or other mechanism by which ceramide levels or function are altered.

Optionally, an active agent is or includes a composition capable of degrading ceramide, optionally selectively degrading ceramide. Illustratively, a composition is or includes a ceramidase, optionally a neutral ceramidase, fragment thereof, derivative thereof, or variant thereof. Optionally, an active agent is a human neutral ceramidase (Pub Med. Accession No: NP_063946). Optionally, a ceramidase is an active fragment, derivative, or variant of human neutral ceramidase. Ceramidase may be obtained from any desired source or organism including but not limited to rat, human, mouse, or other organism. A ceramidase may be isolated from a natural source or recombinantly expressed in a cell system.

Optionally, an active agent is or includes a composition capable inhibiting synthesis of ceramide, optionally selectively inhibiting the function of a neutral sphingomyelinase, optionally a neutral sphingomyelinase. Optionally, an active agent does not alter the function or amount of an acid sphingomyelinase. Any neutral sphingomyelinase inhibitor may be used to varying degrees. Illustrative examples of sphingomyelinase inhibitors include but are not limited to GW4869, Manumycin A, Altenusin, Scyphostatin, C11AG, Macquarimicin A, Cambinol, 4h-1,2,4-triazole-3(2h)-thione derivatives as described in Patent Application Publication WO 2002066447A1, molecules described in European Patent Application Publication EP1207886A2, and antisense oligonucleotides as described in U.S. Pat. No. 9,006,205.

Optionally, an active agent is one that inherently or by the addition of one or more modifiers so as to be incapable of being transported from a peripheral compartment to a non-peripheral compartment as defined herein. Optionally, an active agent excludes a modifier that will promote transport into a neuronal compartment or is administered without a molecule that promotes transport of the active agent into a neuronal compartment.

A compound for treatment or prevention of MDD may be administered in any way suitable and as selected by one of ordinary skill in the art. The active agent may be administered orally, parenterally, intravenously, intramuscularly, intraperitoneally, by transdermal injection, or by contact with a cell or tissue such as by immersion or other form of contact. Injectable or oral dosage forms may be prepared in conventional forms, either liquid solutions or suspensions, solid forms, or as suspension in liquid prior to administration or as emulsions. In some aspects, administration is oral. In some aspects, administration is intravenous.

The dose of the active agent may vary depending on the identity of the active agent and intended route of administration. Furthermore, the composition can be prepared for an administration in a dose of from 0.001 mg/kg body weight to 10 mg/kg body weight, optionally 0.01 mg/kg body weight to 1 mg/kg body weight, optionally 0.1 mg/kg body weight, or be used for such an administration.

In addition or in substitution to neutralization, ceramide is optionally reduced in quantity in a cell, tissue, compartment, or system of an organism. Optionally, the amount of ceramide following administration for a suitable time and a suitable dose may be reduced by 0.001 molar percentage or more, optionally 0.01 molar percentage or more, optionally 0.1 molar percentage or more, optionally 1 molar percentage or more, optionally 5 molar percentage or more, optionally 10 molar percentage or more, optionally 15 molar percentage or more, optionally 20 molar percentage or more, with percent reductions relative to prior to the initiation of the administration to the subject or administration protocol.

In some aspects, the concentration of ceramide following administration of an active agent or stress is at or less than 400% higher than the average concentration from a population of individuals that are not diagnosed with MDD or at risk for MDD in the same tissue or ceramide test source, optionally peripheral blood. Optionally the concentration of ceramide following administration at or less than 300% higher than the average concentration from a population of individuals that are not diagnosed with MDD or at risk for MDD in the same tissue or ceramide test source, optionally at or less than 200% higher, optionally at or less than 100% higher, optionally at or less than 50% higher, optionally at or less than 25% higher.

In some aspects, the concentration of ceramide in the plasma of a subject following administration of an active agent or stress is at or less 8000 pmol/mL. In some aspects, the concentration of ceramide in the plasma of a subject following administration of an active agent or stress is at or less 7000 pmol/mL, optionally 6500 pmol/mL, optionally 6000 pmol/mL, optionally 5500 pmol/mL, optionally 5000 pmol/mL, optionally 4000 pmol/mL.

In another aspect, a method of neutralizing ceramide in the blood of a subject suffering from MDD or at risk for developing MDD is provided, the method comprising administering to the subject an effective amount of an anti-ceramide antibody, whereby ceramide in the blood of the subject is neutralized, thereby treating or reducing the risk of developing MDD in the subject. In a specific embodiment, the anti-ceramide antibody is a humanized antibody.

In another aspect, a method of neutralizing ceramide in the blood of a subject suffering from MDD or at risk for developing MDD is provided, the method comprising administering to the subject an effective amount of an active agent comprising a ceramide-binding domain, whereby ceramide in the blood of the subject is neutralized, thereby treating or reducing the risk of developing MDD in the subject. In a specific embodiment, a peptide or protein active agent comprises a ceramide binding domain selected from the group consisting of an EF-hand calcium-binding motif, a heat shock chaperonin-binding motif (STI1), a sterol-binding domain (SCP2), and a tetratricopeptide repeat region motif.

In another aspect, a method of degrading ceramide in the blood of a subject suffering from MDD or at risk for developing MDD is provided, the method comprising administering to the subject an effective amount of a neutral ceramidase, whereby ceramide in the blood of the subject is degraded, thereby treating or reducing the risk of developing MDD in the subject. In a specific embodiment, the neutral ceramidase is human neutral ceramidase or a fragment, derivative, or variant thereof.

In another aspect, a method of inhibiting ceramide synthesis in the periphery of a subject suffering from MDD or at risk for developing MDD is provided, the method comprising administering to the subject an effective amount of a neutral sphingomyelinase inhibitor, whereby ceramide synthesis in the periphery of the subject is inhibited, thereby treating or reducing the risk of developing MDD in the subject.

Also provided herein are methods of screening candidate agents to treat MDD. In one aspect, a method for identifying a candidate agent to treat MDD is provided, the method comprising: contacting ceramide with a test compound in a first sample; contacting ceramide with a positive control in a second sample; and measuring a concentration of functional ceramide in the first and second samples, wherein a reduction in the concentration of functional ceramide in the first sample compared to the second sample or other control indicates that test compound is a candidate agent for treating MDD. As used herein, “functional ceramide” refers to ceramide that is not deactivated, bound, degraded, or otherwise rendered nonfunctional as a result of binding to the test compound.

In another aspect, a method for identifying a candidate agent to treat MDD is provided, the method comprising: contacting an intact cell capable of synthesizing ceramide with a test compound in a first sample; optionally contacting an intact cell capable of synthesizing ceramide with a control in a second sample or simply allowing the second sample to produce ceramide; and measuring a concentration of ceramide in the first and second samples, wherein a reduction in the concentration of ceramide in the first sample compared to the second sample indicates that the test compound is a candidate agent for treating MDD.

In another aspect, a method for identifying a candidate agent to treat MDD is provided, the method comprising: contacting an intact cell capable of synthesizing ceramide with a test compound in a first sample; contacting an intact cell capable of synthesizing ceramide with a negative control in a second sample; and measuring a concentration of ceramide in the first and second samples, wherein a reduction in the concentration of ceramide in the first sample compared to the second sample indicates that the test compound is a candidate agent for treating MDD.

Optionally, a method of identifying a candidate agent to treat MDD is provided that includes contacting an intact cell capable of synthesizing ceramide with a test compound in a first sample with both an inducer of ceramide synthesis and a test compound, contacting an intact cell capable of synthesizing ceramide with the inducer of ceramide synthesis in a second sample, and measuring a concentration of ceramide in the first and second samples, wherein a reduction in the concentration of ceramide in the first sample compared to the second sample indicates that the test compound is a candidate agent for treating MDD.

Optionally, a method of identifying a candidate agent to treat MDD is provided that includes contacting a subject with a test compound and obtaining a first sample from one or more peripheral compartments of the first subject, measuring a concentration of ceramide in the first sample, optionally measuring the concentration of ceramide in a peripheral compartment of a second subject, wherein a reduction in the concentration of ceramide in the first sample compared to the second sample or standard value indicates that the test compound is a candidate agent for treating MDD. A first and second subject may optionally express elevated levels of ceramide in one or more peripheral compartments relative to a subject that is not stressed or otherwise is not diagnosed with or exhibits characteristic(s) of MDD.

Methods of measuring the concentration of ceramide in a cell or other sample may be any method known in the art, optionally by suing an ELISA using an anti-ceramide antibody for the detection of ceramide, mass spectrometry, kinase assay, immunofluorescence, or any other suitable method, optionally wherein the method of detection is suitable for quantification of ceramide.

Optionally, a method of identifying a candidate agent to treat MDD is provided that includes contacting a first subject with a test compound and subjecting the first subject to one or more physical or emotional tests for the presence or absence of MDD, optionally subjecting a second subject to one or more physical or emotional tests for the presence or absence of MDD wherein alteration in the value of the one or more physical or emotional tests in the first subject relative to the second subject or standard value indicates that the test compound is a candidate agent for treating MDD. A first and second subject may optionally express elevated levels of ceramide in one or more peripheral compartments relative to a subject that is not stressed or otherwise is not diagnosed with or exhibits characteristic(s) of MDD. A first or second subject may optionally be clinically diagnosed with MDD or express one or more characteristics of MDD such as in a Hamilton Depression score, or may exhibit one or more abnormal physical or emotional test results such as time outside a box, latency to fee, time in center, splash test result, forced swim test, or coat test, or any combination thereof.

Suitable controls for use in the screening assays disclosed herein include, but are not limited to, neutral sphingomyelinase inhibitors or ceramide synthase inhibitors such as myriocin.

### EXAMPLES

Various aspects of the present disclosure are illustrated by the following non-limiting examples. The examples are for illustrative purposes and are not a limitation on any practice of the present disclosure. It will be understood that variations and modifications can be made without departing from the spirit and scope of the disclosure.

### Example 1. Materials and Methods

### Patients

Depressive patients (7 women, 10 men) aged ≥60 years (mean age=71.1 years, SD=4.7) were recruited at the Psychiatric University Hospital, Basel, Switzerland. All patients had a score >6/15 on the Geriatric Depression Scale (Yesavage, et al., Development and validation of a geriatric depression screening scale: a preliminary report, J. Psychiatr. Res. 17:37-49 (1982-1983)), a score >10/53 on the Hamilton Rating Scale for Depression (Hamilton, A rating scale for depression, J. Neurol. Neurosurg. Psychiatry 23: 56-62 (1960); Williams, A structured interview guide for the Hamilton Depression Rating Scale, Arch. Gen. Psychiatry 45: 742-747 (1988)) and a score >16/63 on the Beck Depression Inventory (Hautzinger, et al., Harcourt Test Services, Frankfurt (2007)). For comparison, 9 healthy individuals without history of depression (mean age=70.9 years, SD=5.3) were included in the study. The study was approved by the “Ethikkommission der Nordwest- and Zentralschweiz” (EKNZ; No. 2015-148) and all participants provided written informed consent.

At baseline and after remission of depressive symptoms (with a therapy regimen consisting of interpersonal psychotherapy (IPT-late-life) and an individual psychopharmacotherapy with antidepressants/mood stabilizers (using citalopram, escitalopram, venlafaxine, duloxetine, mirtazapine, agomelatine, bupropion, vortioxetine, trazodone, valproic acid, aripiprazole, quetiapine, lithium)), venous blood draws were performed in all participants in the morning. In addition, samples from patients included in the “Ceramide-associated Biomarkers in Depression” study approved by the Ethics Committee of the Medical Faculty of the Friedrich-Alexander University Erlangen-Nürnberg (FAU, ID 148_13 B, 17 Jul. 2013) were included (Wagner C J, Musenbichler C, Böhm L, Färber K, Fischer A-I, von Nippold F, Winkelmann M, Mühle C, Kornhuber J, Lenz B: LDL cholesterol relates to depression, its severity, and the prospective course. Progress in Neuro-Psychopharmacology & Biological Psychiatry 2019. Mühle C, Wagner CJ, Färber K, Richter-Schmidinger T, Gulbins E, Lenz B, Kornhuber J: Secretory acid sphingomyelinase in the serum of medicated patients predicts the prospective course of depression. Journal of Clinical Medicine 2019; 8.)

### Mice and Treatments

Corticosterone (Sigma, St. Louis, Mo.) was administered to mice via the drinking water at 120 mg/L for 7 days. In the chronic unpredictable stress model (CUS), the mice were challenged with unpredictable environmental stress for 8 days, i.e., a reversal of the light/dark cycle, 3 h of 45° tilting of the cage twice each week, shaking at 125 rpm, food deprivation for 14 h, predator sounds (15 min) or wet cages (1 h) with two forms of stress per day in a randomized (unpredictable) order. Blood was collected from the heart into heparin-coated needles and tubes immediately after sacrifice by cervical dislocation. Blood plasma was centrifuged at 3500 rpm for 5 min at 4° C. in an Eppendorf centrifuge. The plasma was carefully removed and shock frozen in liquid nitrogen.

Embryonic stem cells allowing inducible deletion of neutral sphingomyelinase 2 (Smpd3) were obtained from the European Mouse Mutant Cell Repository (Helmholtz Center Munich, Germany). The gene symbol of the mice is Smpd3^{tm1a(EUCOMM)Hmg}. Transgenic mice were generated, the Flp site was deleted resulting in a mouse strain in which exon 2 of Smpd3 is flanked by two lox sites. The mice were crossed with Cre-Er mice (Taconic, Cologne, Germany) and deletion of exon 2 was induced by 3 intraperitoneal injections (every second day) of 100 μg/25 g body weight tamoxifen.

Bromodeoxyuridine (BrdU, 2 mg/25 g body weight) was injected 3-times, once every 2 h, at a dose of 75 mg/kg, starting 16 h before the mice were sacrificed.

Anti-ceramide antibodies (2 μg/25 g body weight; purified monoclonal mouse anti-ceramide IgM clone S58-9 (Glycobiotech, Kuekel, Germany), monoclonal mouse anti-ceramide IgG (Catalogue #111583, Antibody Research Corp., St. Peters, Mo.), and a monoclonal mouse anti-ceramide IgM clone MID15B4 (Product No. C8104, Sigma Aldrich, St. Louis, Mo.)), a control IgM (2 μg/25 g body weight) and recombinant neutral ceramidase (1 μg/25 g body weight; Asah2, R&D, 3,000 pmol/min/m) were intravenously injected for in vivo treatment of depressed mice.

To achieve in vitro neutralization of consumption of ceramide, 125 μL blood plasma or exosomes (purified from 150 μL blood plasma as described below) were incubated with 0.5 μg purified IgM anti-ceramide antibodies, 0.5 μg control IgM or 1 μg recombinant neutral ceramidase.

### Measurement of Ceramide by Kinase Assay

10 μL of blood plasma or of resuspended exosomes (corresponding to 50 μL plasma) was added to 190 μL H₂O and extracted in 600 μL CHCl₃:CH₃OH:1N HCl (100:100:1, v/v/v). The lower phase was collected, dried, resuspended in 20 μL of a detergent solution (7.5% [w/v] n-octyl glucopyranoside, 5 mM cardiolipin in 1 mM diethylenetriamine-pentaacetic acid [DTPA]), and micelles were obtained by bath sonication for 10 min. The kinase reaction was initiated by the addition of 70 μL of a reaction mixture containing 10 μL diacylglycerol (DAG) kinase (GE Healthcare Europe, Munich, Germany), 0.1 M imidazole/HCl (pH 6.6), 0.2 mM DTPA, 70 mM NaCl, 17 mM MgCl₂, 1.4 mM ethylene glycol tetraacetic acid, 2 mM dithiothreitol, 1 μM adenosine triphosphate (ATP), and 5 μCi [³²P]γATP. The kinase reaction was performed for 60 min at room temperature with shaking at 300 rpm. The assay was terminated by the addition of 1 mL CHCl₃:CH₃OH:1N HCl (100:100:1, v/v/v), 170 μL buffered saline solution (135 mM NaCl, 1.5 mM CaCl₂), 0.5 mM MgCl₂, 5.6 mM glucose, 10 mM HEPES, pH 7.2), and 30 μL of a 100-mM ethylenediaminetetraacetic acid (EDTA) solution. The samples were vortexed, phases were separated, and the lower phase was collected. The samples were then dried, separated on Silica G60 thin-layer chromatography (TLC) plates with chloroform/acetone/methanol/acetic acid/H₂O (50:20:15:10:5, v/v/v/v/v), and developed with a Fuji phosphorimager. Ceramide amounts were determined by comparison with a standard curve; C₁₆ to C₂₄ ceramides were used as substrates.

### Ceramide Measurements by Mass Spectrometry

Samples were subjected to lipid extraction with 1.5 mL methanol/chloroform (2:1, v:v). The extraction solvent contained C₁₇-ceramide and C₁₆-d₃₁-sphingomyelin (both Avanti Polar Lipids, Alabaster, Ala., USA) as internal standards. Extraction was facilitated by incubation at 48° C. with gentle shaking (120 rpm) overnight. After lipid extraction, samples were saponified with 150 μL 1 M methanolic KOH for 2 h at 37° C. with gentle shaking (120 rpm). Samples were then neutralized with 12 μL glacial acetic acid and centrifuged at 2,200×g for 10 min at 4° C. Organic supernatants were evaporated to dryness by vacuum centrifugation with a Savant SpeedVac concentrator (Thermo Fisher Scientific, Dreieich, Germany). Dried residues were reconstituted in 200 μL of a 95:5 (v:v) mixture of high-performance liquid chromatography (HPLC) eluents B:A (see below), thoroughly vortexed for 10 min at 1,500 rpm, centrifuged at 2,200×g for 10 min at 4° C., and subjected to mass spectrometric sphingolipid quantification. All analyses were conducted with a 1200 series high-performance liquid chromatograph coupled to a quadrupole time-of-flight (QTOF) 6530 mass spectrometer (Agilent Technologies, Waldbronn, Germany) operating in the positive electrospray ionization (ESI+) mode.

Chromatographic separations were achieved on a ZORBAX Eclipse Plus C8 column (2.1×150 mm, 3.5 μm; Agilent Technologies) at 30° C. The injection volume per sample was 10 μL. A mobile phase system consisting of water (eluent A) and acetonitrile/methanol (1:1, v:v; eluent B), both acidified with 0.1% formic acid, was used for gradient elution at an initial composition of 10:90 (A:B, v:v) and a flow rate of 0.7 mL/min. The total run time for one analysis, including re-equilibration of the HPLC system, was 34 min. For mass spectrometric measurements, the following ion source settings were adjusted: sheath gas temperature, 380° C.; sheath gas flow, 12 L min⁻¹ of nitrogen; nebulizer pressure, 45 psig; drying gas temperature, 360° C.; drying gas flow, 10 L min′ of nitrogen; capillary voltage, 4500 V; fragmentor voltage, 155 V; and nozzle voltage, 2000 V. Ceramides were analyzed in tandem mass spectrometry (MS/MS) mode using the fragmentation of the precursor ions into the product ion m/z 264.270 for ceramides. A collision energy of 25 eV was applied for collision-induced dissociation (CID). Quantification was performed by external calibration with MassHunter software (Agilent Technologies). Calibration curves of reference ceramides were performed from 1 pmol to 100 pmol per injection and were constructed by linear fitting using the least-squares linear regression calculation. The resulting slope of the calibration curve was used to calculate the concentration of the respective analyte in the samples. Determined ceramide amounts were normalized to protein.

### Exosomes

To isolate exosomes from blood plasma, samples were centrifuged at 16,500×g for 20 min. The supernatant was then passed through a 0.22-μm filter and exosomes were harvested by centrifugation twice at 100,000×g for 70 min. The final pellet was resuspended in phosphate-buffered saline (PBS). Exosomes were pelleted by centrifugation at 100,000×g for 70 min, the supernatant was discarded, and the exosomes were resuspended and injected i.v. into healthy mice.

Loading of Plasma and Exosomes with C16 Ceramide or Deuterated C16 Ceramide

Plasma or exosomes were loaded with C₁₆ ceramide or deuterated C₁₆ ceramide (Avanti Polar Lipids, USA) by incubation of purified exosomes with 2 μM C₁₆ ceramide or 2 μM deuterated C₁₆ ceramide in 100 μL PBS for 60 min at 37° C. Plasma samples were directly injected i.v. into healthy mice. Exosomes were centrifuged again at 100,000×g for 70 min, the supernatant was discarded, resuspended in PBS and injected i.v. into healthy mice.

### Immunohistochemical Bromodeoxyuridine Staining

For BrdU staining, mice were injected with BrdU 4 times, once every 2 h. Mice were sacrificed 24 h after the first injection and brains were prepared as above. Acetone-fixed samples were treated with pepsin for 20 min at 37° C., washed, incubated at 65° C. for 2 h with 50% formamide in 300 mM NaCl and 30 mM sodium citrate (pH 7.0), and washed twice in sodium citrate buffer. The DNA was denatured for 30 min at 37° C. with 2 M HCl, washed, neutralized for 10 min with 0.1 M borate buffer (pH 8.5), washed, and blocked with 0.05% Tween 20 and 5% FCS in PBS (pH 7.4). The samples were then stained for 45 min at 22° C. with 5 μg/mL BrdU-specific antibodies (Roche, Mannheim, Germany, #111703760001), washed, and stained with Cy3-coupled F(ab)₂ anti-mouse IgG antibody fragments (Jackson ImmunoResearch, West Grove, Pa.).

All sections were analyzed with a LEICA TCS SL fluorescence confocal microscope. Every tenth section of serial sections of the hippocampus was counted by an investigator blinded to the nature of the samples.

### Behavioral Studies

Behavioral testing was performed between 3:00 p.m. and 6:00 p.m. under diffuse indirect room light. All tests were performed on separate days. If appropriate, animals were tracked with a video camera (Noldus Systems, Worpswede, Germany). For the novelty-suppressed feeding test (latency to feed), mice were fasted for 24 h. The time was recorded during which the mice explored a new environment before they began eating. For the light/dark box test, mice were placed in a dark and safe compartment that was connected via a 5-cm×5-cm rounded-corner aperture to an illuminated, open, and thus aversive area. The time that the mouse spent in each of the separate compartments was recorded. In the open-field arena test (time in center), the mice were released near the wall of a 50-cm×50-cm white plastic cage with sidewalls 30 cm high. Animals were observed for 30 min, and the time during which the animal was more than 10 cm away from the wall was recorded. In the coat state test, the appearance of the coat (groomed vs unkempt coat) was scored on the head, neck, back, and ventrum with either a zero for normal status or a 1 for unkempt status. For the forced swim test, mice were placed in a cylinder filled with water (21-23° C.) for 15 min. After 24 h the mice were again placed in a water-filled cylinder for 6 min, and the time of mobility or immobility during the last 4 min of the second trial was recorded. Mice were judged immobile when they moved only to keep their heads above water.

### Quantification and Statistical Analysis

Data are expressed as arithmetic means±SD. For the comparison of continuous variables from independent groups with one variable (treatment), one-way ANOVA was used, followed by post-hoc Student's t-tests for all pairwise comparisons, applying the Bonferroni correction for multiple testing. The P values for the pairwise comparisons were calculated after Bonferroni correction. All values were normally distributed, and the variances were similar. For the analysis of groups with 2 variables (treatment and genotype) two-way ANOVA was used. Statistical significance was set at a P value of 0.05 or lower (two-tailed). The sample size planning was based on the results of two-sided Wilcoxon-Mann-Whitney tests (free software: G*Power, Version 3.1.7, University of Duesseldorf, Germany). Investigators were blinded to results of histologic analyses and to animal identity. Before the experiments, animals were randomly assigned to cages by a technician who was not involved in the experiments. Cages were randomly assigned to the various experimental groups. Western blots were quantified with Image J software (National Institutes of Health, Bethesda, Md., USA), and results are expressed as arbitrary units (a.u.).

### Example 2. Ceramide Levels in Blood Plasma and Exosomes are Higher in Mice and Humans with MDD than in Control Subjects

Although it is known that major depressive disorder is often caused by stress, the mechanism by which stress induces MDD is unknown. The present investigators investigated the hypothesis that exogenous stress induces the release of ceramide-enriched exosomes into the blood.

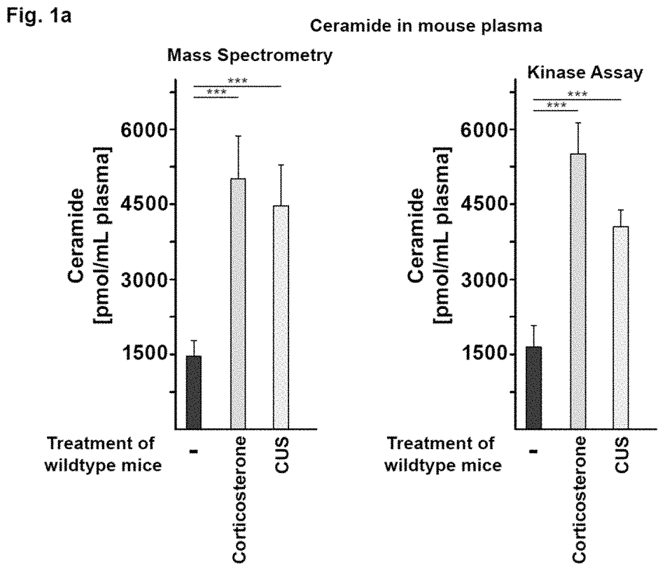

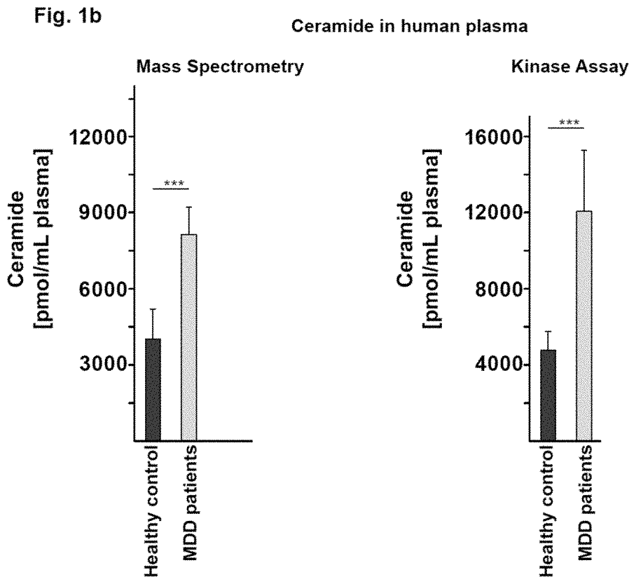

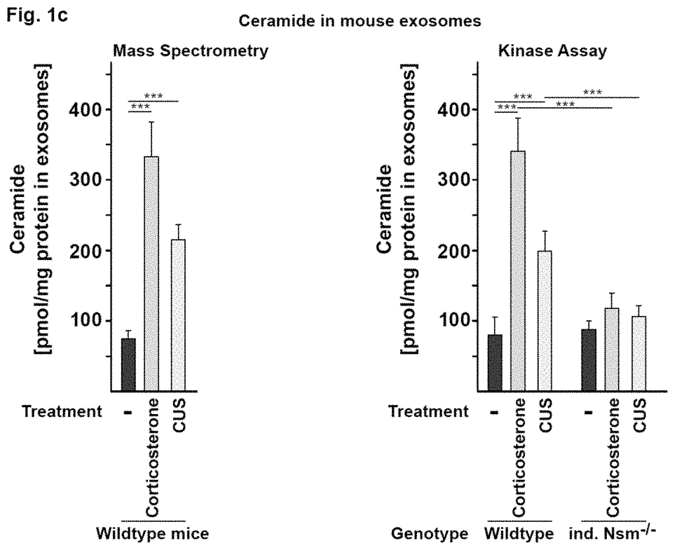

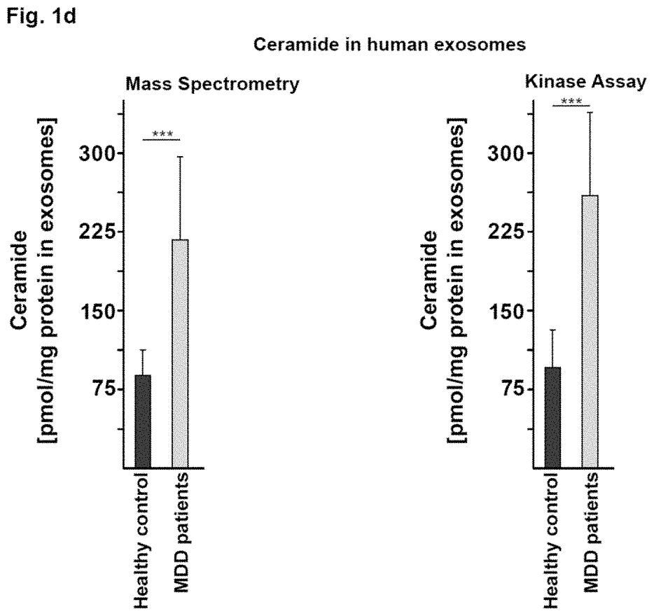

Wildtype mice or mice deficient in neutral sphingomyelinase 2 were stressed with either glucocorticosterone or chronic unpredictable environmental stress (CUS) or were left untreated as controls. Venous blood samples were collected and blood plasma was prepared. Blood plasma was also prepared from patients with major depressive disorder (MDD) and from healthy control subjects. In addition, exosomes were purified from murine and human blood plasma by ultracentrifugation. Ceramide concentrations were determined by mass spectrometry and ceramide kinase assays. FIGS. 1a-1d show the mean±SD, n=6 for all mouse samples and n=16 for all human samples. *P<0.05, **P<0.01, ***P<0.001, ANOVA.

The results of these studies demonstrate that stress induces a marked increase of ceramide levels in the blood plasma of wildtype mice (FIG. 1a). The analysis of blood samples from patients with MDD showed a similar increase in ceramide concentrations in the plasma of patients with MDD compared to healthy individuals (FIG. 1b).

Because ceramide is a very hydrophobic molecule, the investigators examined whether ceramide is released into the blood plasma within exosomes upon the application of various forms of stress. Exosomes were purified from blood plasma taken from stressed or non-stressed mice, from patients with MDD, or from healthy control subjects and then ceramide concentrations were determined in these exosomal preparations. The results indicate that glucocorticosterone-mediated or chronic unpredictable environmental stress induces a marked increase in ceramide concentrations in exosomes in the blood plasma of mice (FIG. 1c).

Neutral sphingomyelinases have previously been shown to be crucial for the release of exosomes and could mediate the release of ceramide within exosomes. To test the role of the neutral sphingomyelinases in stress responses, the investigators used mice with an inducible deletion of neutral sphingomyelinase 2 (deletion of the neutral sphingomyelinase 2 was induced by Cre-recombination in adult mice). The results demonstrated that ceramide concentrations in the exosomal fraction of mice deficient in neutral sphingomyelinase 2 do not increase, whereas wildtype littermates respond to stress with an increased concentration of ceramide in exosomes (FIG. 1c).

Similarly, results showed that the ceramide concentration in human exosomes purified from the plasma of MDD patients is approximately 3-times higher than that in exosomes from the plasma of healthy control subjects (FIG. 1d).

### Example 3. Neutralization or Consumption of Ceramide in the Blood Plasma Prevents MDD in Mice

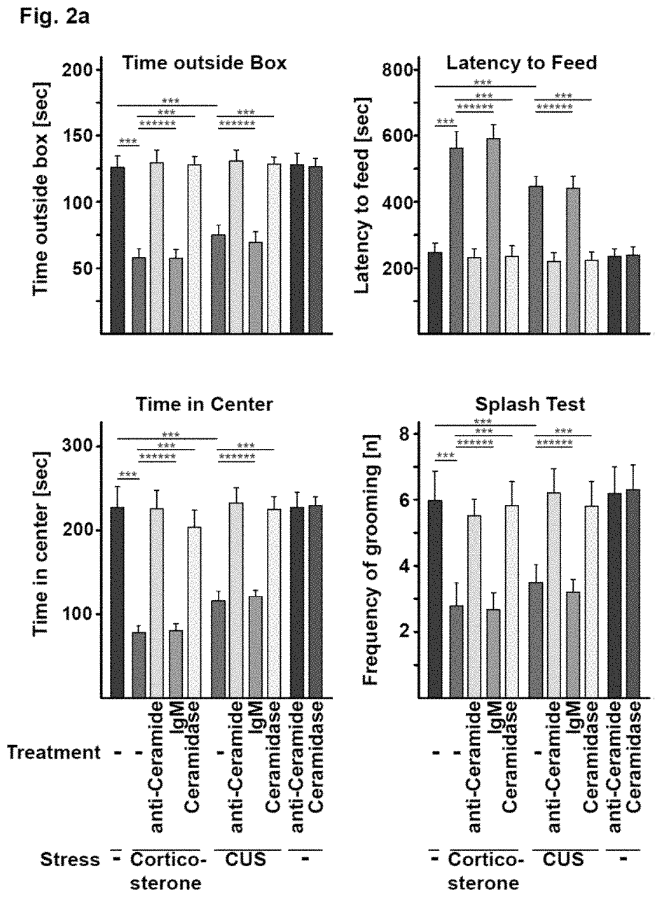

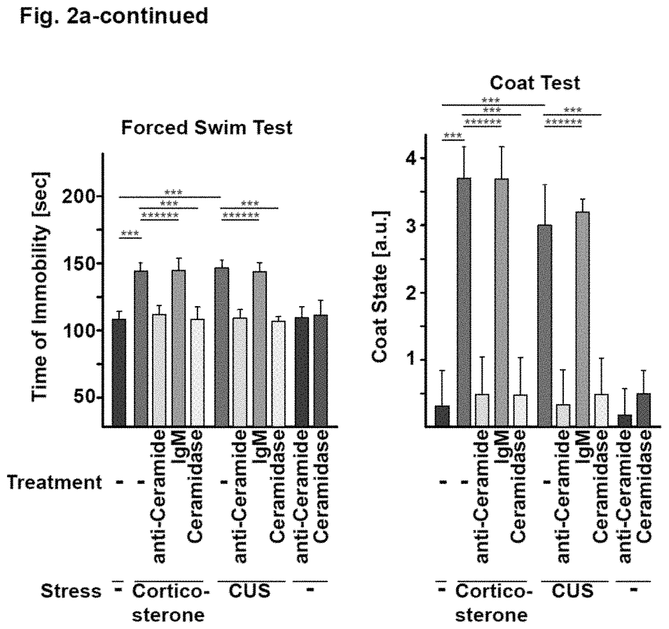

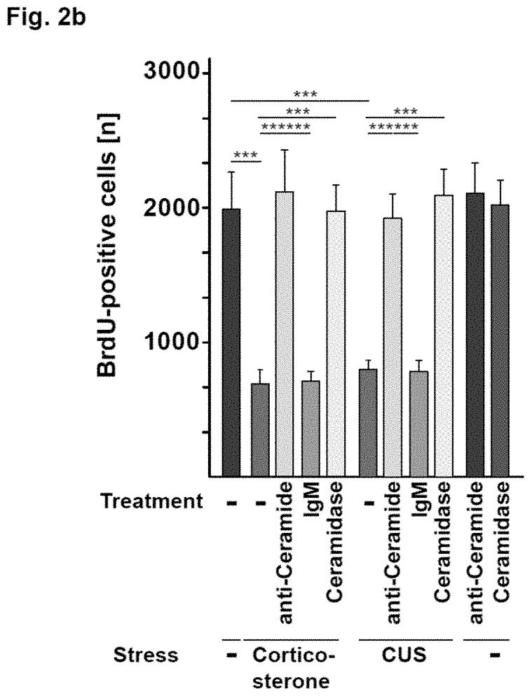

The biomedical importance of the increase in ceramide concentrations in the blood plasma was investigated. 24 h and 12 h before analysis, mice that had already been exposed to glucocorticosterone or chronic unpredictable environmental stress (CUS) for 6 days or left untreated were injected with (i) anti-ceramide IgM antibodies (purified monoclonal mouse anti-ceramide IgM clone S58-9 (Glycobiotech, Kuekel, Germany) to neutralize ceramide in the plasma or with (ii) recombinant neutral ceramidase to consume plasma ceramide. Controls were not stressed and left untreated or received i.v. injection of anti-ceramide antibody or ceramidase. Because it was proposed that ceramide induces acute changes in the brain, investigators determined behavior and neuronal proliferation 24 h after the first injection of anti-ceramide antibody or ceramidase. Neurogenesis in the hippocampus 24 h after treatment was determined by staining for 5′-bromo-2′-deoxyuridine (BrdU). FIGS. 2a-2b show the mean±SD from each 6 samples. *P<0.05, **P<0.01, ***P<0.001, ANOVA.

Although the pathogenetic role of neuronal proliferation in MDD is not yet settled, but neurogenesis is clearly reduced in stress and is at least a good marker for the effects of antidepressants. The results show that neutralizing or consuming ceramide with anti-ceramide antibodies or ceramidase abrogated behavioral signs of major depression in stressed mice and normalized neurogenesis in these mice (FIGS. 2a, 2b). Injecting control IgM exerted no effect on stress-induced major depression (FIGS. 2a, 2b).

These findings indicate that the release of ceramide into the blood upon the application of stress is crucial for the induction of MDD.

A similar study was carried out with two additional anti-ceramide antibodies: a monoclonal mouse anti-ceramide IgG (Catalogue #111583, Antibody Research Corp., St. Peters, Mo.), and a monoclonal mouse anti-ceramide IgM (Product No. C8104, clone MID15B4, Sigma Aldrich, St. Louis, Mo.).

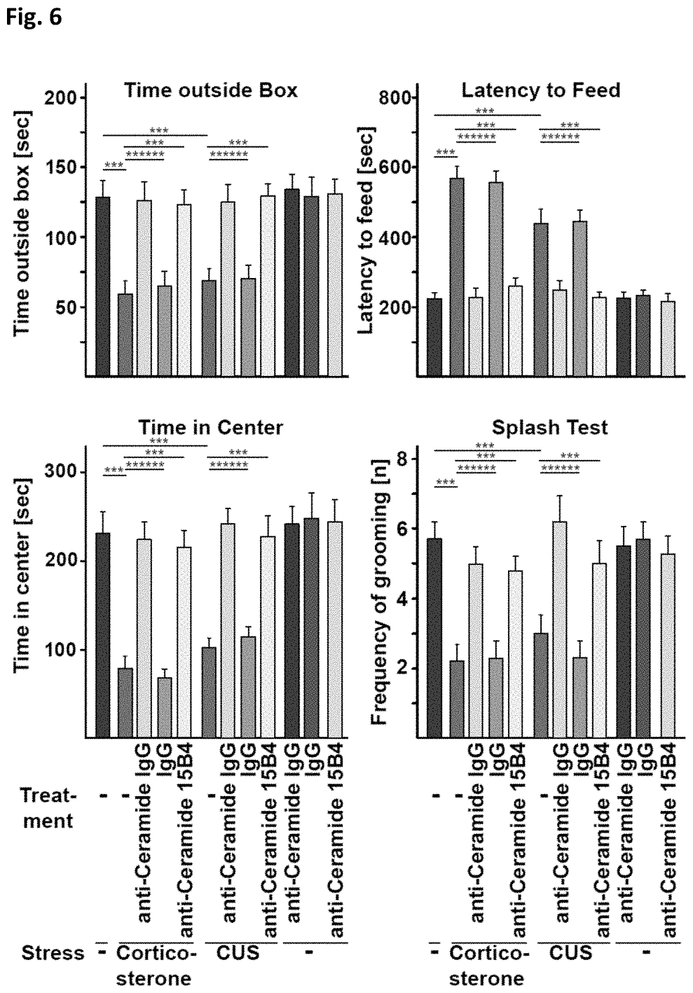

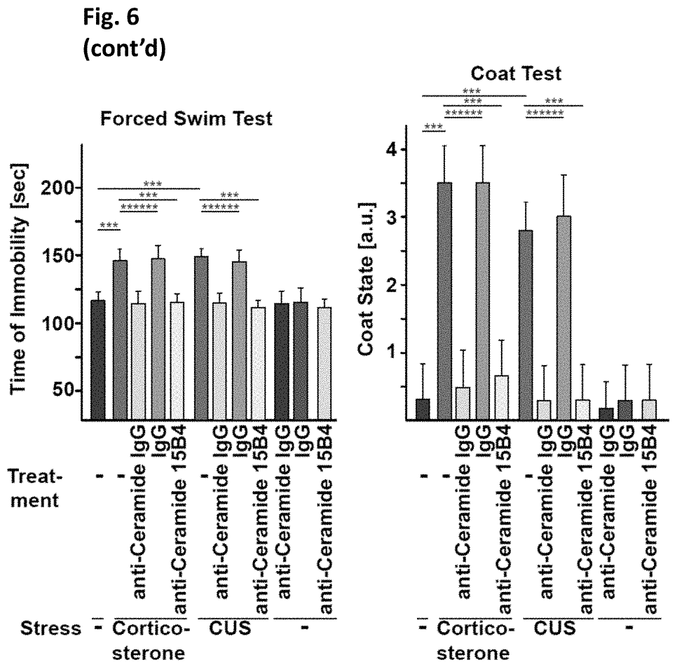

Wildtype mice were stressed for 6 days with either glucocorticosterone or chronic unpredictable environmental stress (CUS) or were left untreated. On day 5, anti-ceramide IgG or monoclonal anti-ceramide 15B4 antibodies or control immunoglobulin G (IgG) were i.v. injected. Controls were not stressed and left untreated or received i.v. injection of anti-ceramide antibodies. Behavioral changes were then analyzed 24 h later by the light/dark box test, the novelty-suppressed feeding test, the open-field arena test, the splash test, the forced swim test and the coat status. FIG. 6 shows the mean±SD from each 6 samples. *P<0.05, **P<0.01, ***P<0.001, ANOVA.

The results after treatment with monoclonal mouse anti-ceramide IgG and a monoclonal mouse anti-ceramide IgM were similar to the original results and indicate that i.v. injection of each anti-ceramide antibody abrogated behavioral signs of major depression in mice stressed by glucocorticosterone or chronic unpredictable environmental stress (FIG. 6). Isotype control IgG antibodies were without effect.

### Example 4. Injection of Plasma or Exosomes from Stressed Subjects to Unstressed Subjects Transfers Symptoms of MDD and Ex Vivo Anti-Ceramide Agent Abrogates the Same Effect

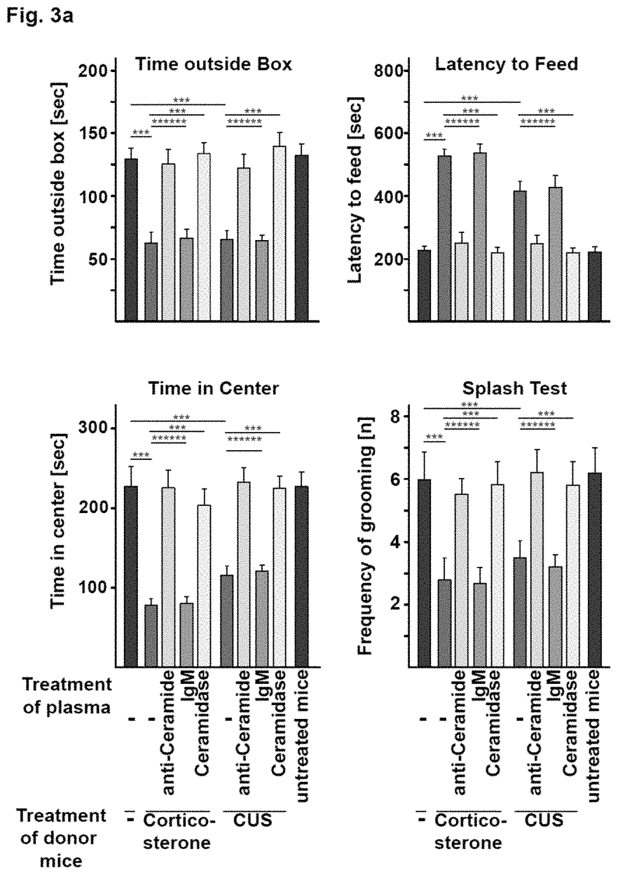

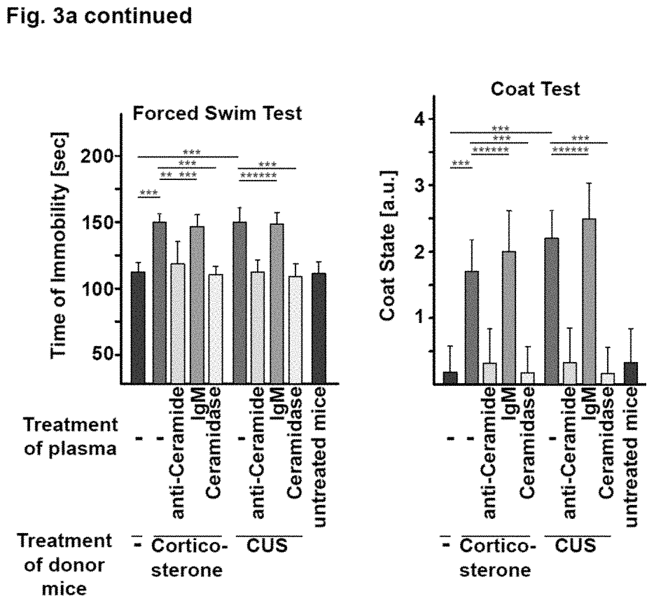

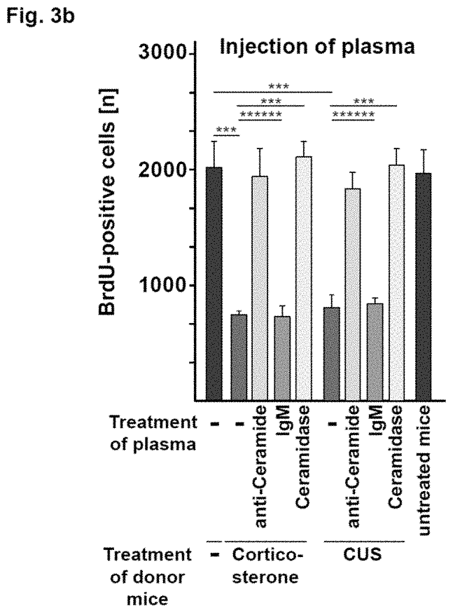

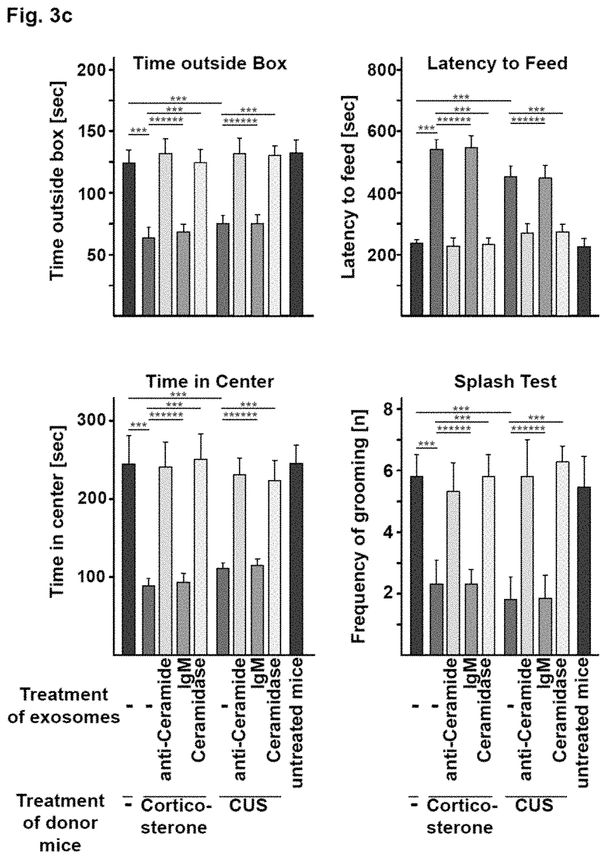

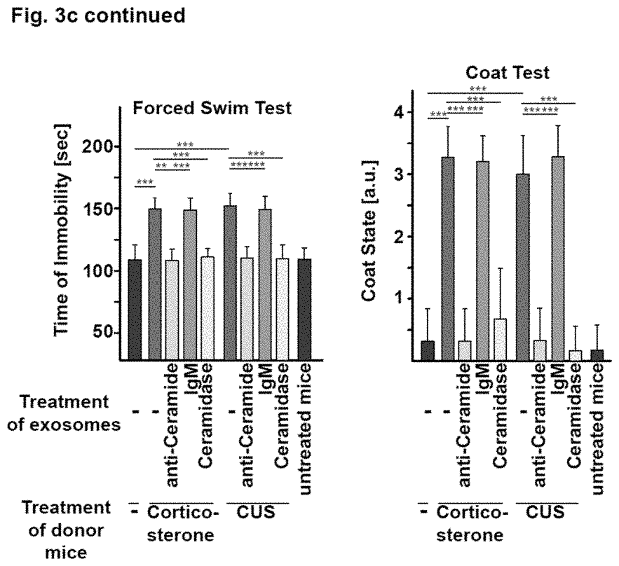

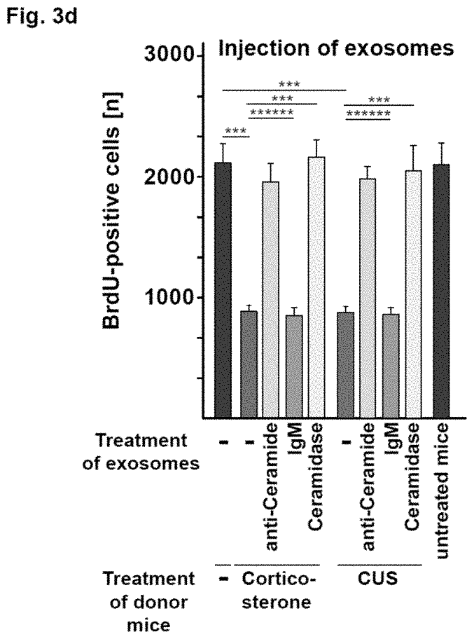

Wildtype mice were stressed with either glucocorticosterone or chronic unpredictable environmental stress (CUS) or were left untreated. Blood plasma or exosomes that were isolated from 150 μL blood plasma, were treated in vitro with anti-ceramide IgM antibodies clone S58-9, control immunoglobulin M (IgM), or recombinant ceramidase or was left untreated. 125 μL of the blood plasma samples or purified exosomes were injected intravenously (i.v.) into healthy wildtype mice, and behavioral changes and neurogenesis in the hippocampus were determined 24 h later as readouts for MDD. FIGS. 3a-3d show the mean±SD from each 6 samples. *P<0.05, **<0.01, ***P<0.001, ANOVA.

The results show, first, that injection of blood plasma from stressed mice into non-stressed, healthy mice transferred the symptoms of MDD (FIGS. 3a, 3b). Second, in vitro incubation of plasma from stressed mice with anti-ceramide antibodies clone S58-9 or ceramidase prior to re-injection into non-stressed mice prevented the development of MDD, whereas control IgM exerted no effect (FIGS. 3a, 3b).

To exclude the possibility that investigators were measuring the effects of corticosterone that might have remained in the plasma of these mice, untreated mice were injected with a high dose of corticosterone (1 mg/kg) and the presence of biochemical and behavioral symptoms of major depression after 24 h were assessed. These studies indicate that treatment with glucocorticosterone for 24 h is not sufficient to induce MDD (data not shown).

To confirm the role of peripheral ceramide in the development of MDD, exosomes were purified from mice that were exposed to glucocorticosterone or chronic unpredictable environmental stress or left untreated. The purified exosomes were incubated with anti-ceramide antibodies clone S58-9, ceramidase, or IgM ex vivo or the samples were left untreated. After an incubation time of 60 min, the exosomes were separated from any unbound antibody or ceramidase by ultracentrifugation and injected intravenously (i.v.) into untreated wildtype. As indicators of MDD investigators again determined behavior parameters and neuronal proliferation. The results show that exosomes purified from stressed mice reduced neuronal proliferation and induce depressed behavior within 24 h (FIGS. 3c, 3d). In vitro incubation of exosomes with anti-ceramide antibodies clone S58-9 or ceramidase prevented development of MDD (FIGS. 3c, 3d). Control IgM did not affect the induction of MDD by exosomes from stressed animals (FIGS. 3c, 3d). Exosomes from untreated animals exerted no effect (FIGS. 3c, 3d).

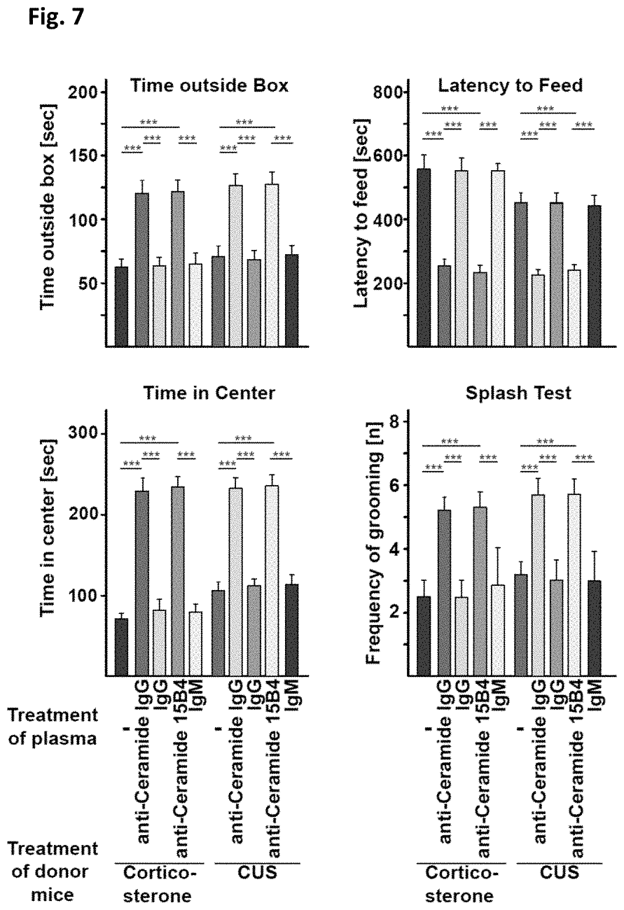

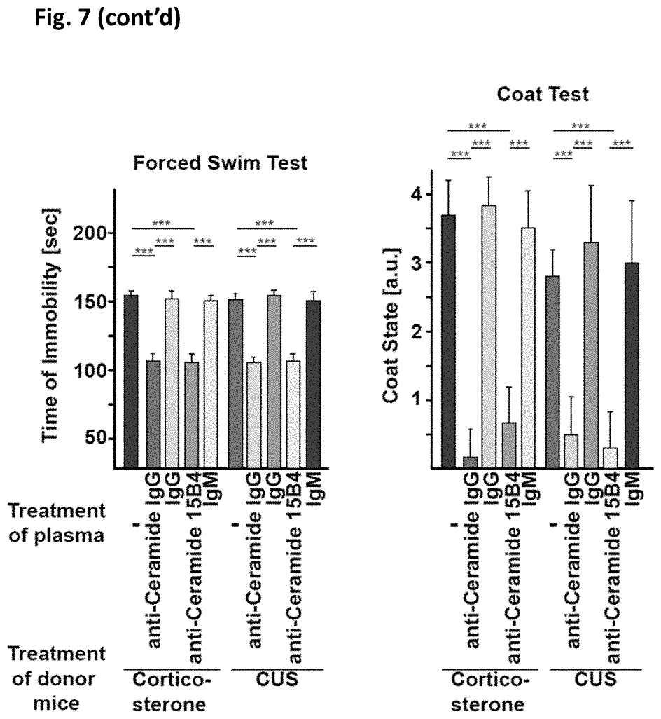

To further confirm results, a similar study was carried out with additional anti-ceramide antibodies. Wildtype mice were stressed with either glucocorticosterone or chronic unpredictable environmental stress (CUS) or were left untreated and blood plasma was obtained from these mice. Blood plasma was treated in vitro with monoclonal mouse anti-ceramide IgG (Catalogue #111583, Antibody Research Corp., St. Peters, Mo.), monoclonal mouse anti-ceramide IgM (Product No. C8104, clone MID15B4, Sigma Aldrich, St. Louis, Mo.), control immunoglobulin G (IgG), control IgM, or was left untreated. 125 μL of the blood plasma samples were injected intravenously (i.v.) into healthy wildtype mice, and behavioral changes and were determined 24 h later as readouts for MDD. FIG. 7 shows the mean±SD from each 6 samples. *P<0.05, **P<0.01, ***P<0.001, ANOVA.

Results show in vitro incubation of blood plasma from stressed mice with two additional, different anti-ceramide antibodies also prevented the induction of behavioral signs of major depression in mice stressed by glucocorticosterone or chronic unpredictable environmental stress (FIG. 7), while the isotype control IgG or IgM antibodies did not affect the induction of depressive behavior upon injection of plasma obtained from stressed mice.

### Example 5. Exosomal Ceramide Determines the Pathogenesis of MDD

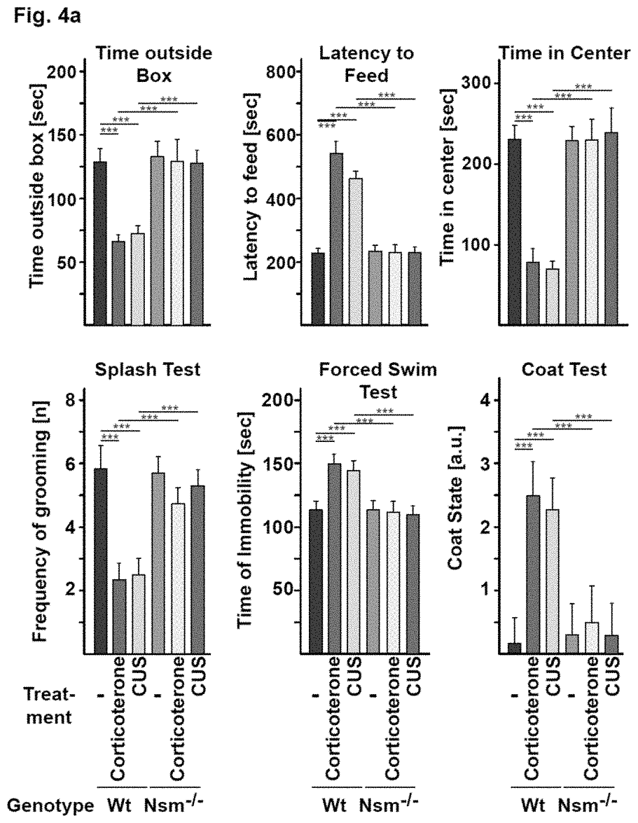

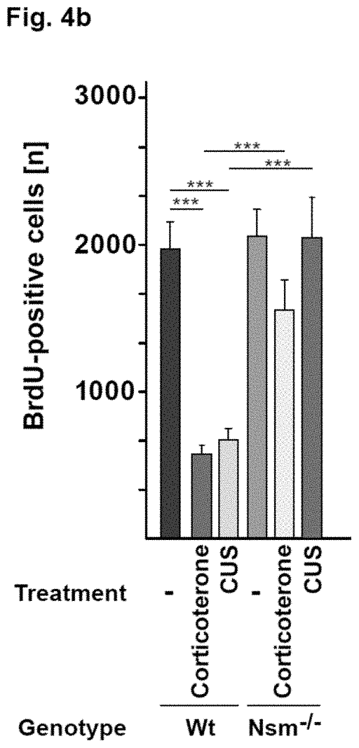

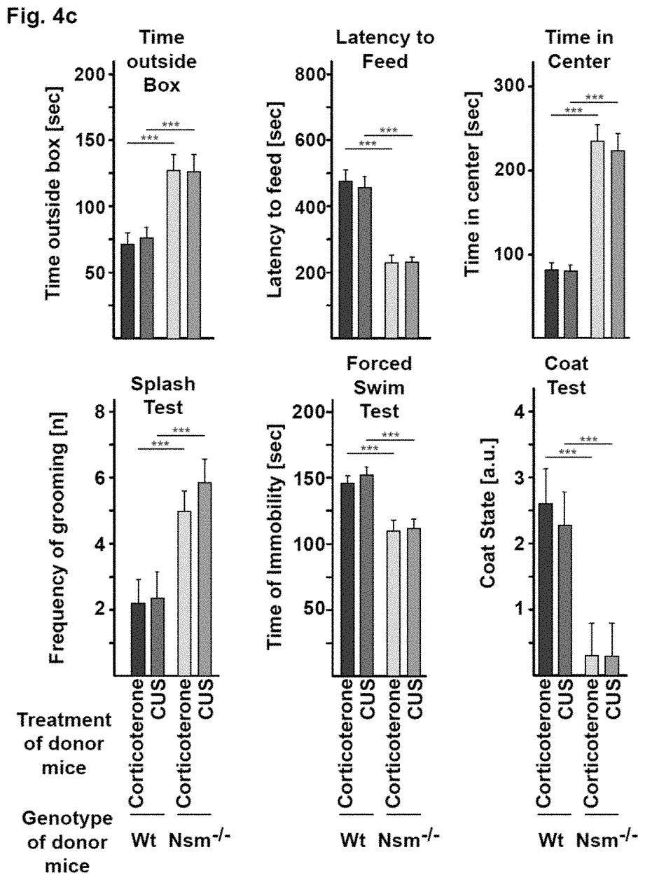

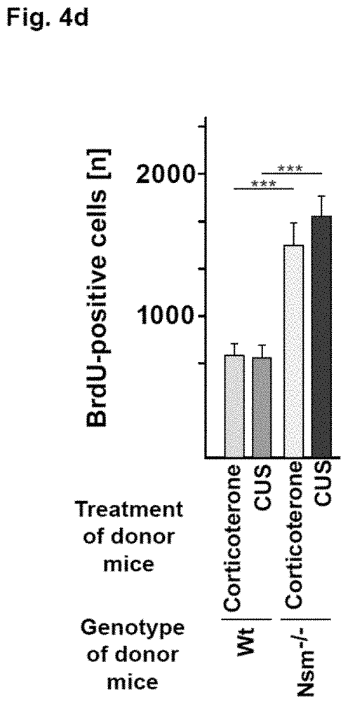

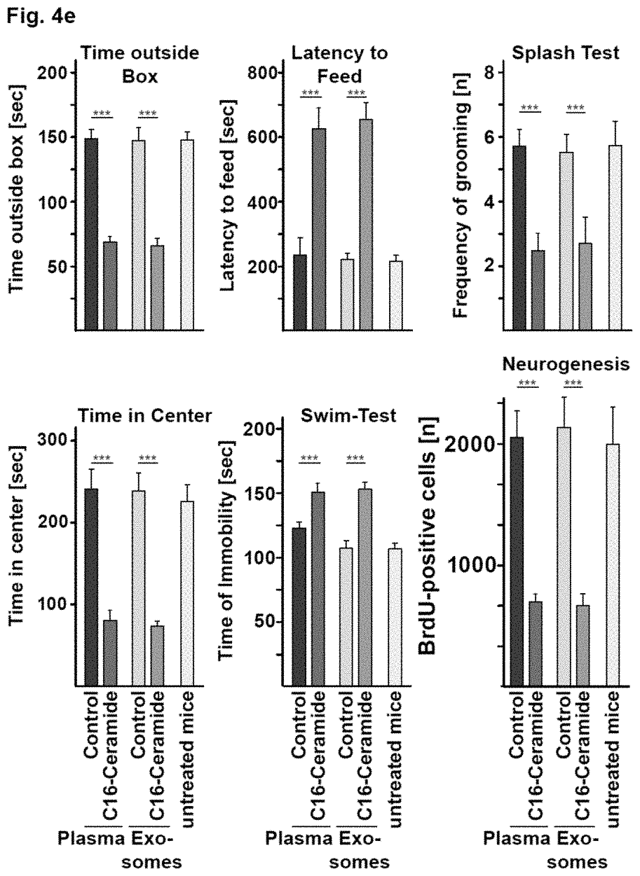

As described above, the increase of ceramide concentrations in the blood of stressed mice requires the expression of neutral sphingomyelinase 2. To confirm the role of the neutral sphingomyelinase 2 for the pathogenesis of MDD, mice deficient in neutral sphingomyelinase 2 and control mice were stressed with either glucocorticosterone or chronic unpredictable environmental stress (CUS) or were left untreated. In addition, exosomes were isolated from 150 μL blood plasma from wildtype mice or mice deficient in neutral sphingomyelinase 2 and were injected intravenously (i.v.) into healthy wildtype mice. Behavioral changes and neurogenesis were measured to analyze major depressive disorder. Displayed are the mean±SD from each 6 samples. For FIGS. 4a-4d, *P<0.05, **P<0.01, ***P<0.001, ANOVA.

The results show various forms of stress did not induce changes in behavior and only slightly reduced neurogenesis in mice deficient in neutral sphingomyelinase 2, whereas they exerted strong effects in wildtype littermates (FIGS. 4a, 4b). In accordance, injecting plasma or exosomes isolated from stressed mice deficient in neutral sphingomyelinase 2 into untreated wildtype mice did not induce any signs of major depression, whereas injecting exosomes from wildtype littermates induced MDD within 24 h (FIGS. 4c, 4d).

Next, investigators tested whether MDD can be induced in untreated mice by increasing plasma concentrations of ceramide. Plasma or exosomes were isolated from the blood of untreated wildtype mice, loaded with 2 μM C16-ceramide and injected intravenously (i.v.) into wildtype mice. Behavioral changes and neurogenesis were measured as readout for the induction of MDD by i.v. injected C₁₆-ceramide loaded plasma or exosomes. FIG. 4e shows the mean±SD from each 6 samples. *P<0.05, **P<0.01, ***P<0.001, ANOVA.

These experiments showed that loading the blood plasma or exosomes with C₁₆-ceramide is sufficient to induce MDD within 24 h as determined by behavioral changes and neuronal proliferation (FIG. 4e).

### Example 6. Injection of Exosomes Isolated from the Blood Plasma of Patients with MDD Induces Depressive Behavior in Healthy Wildtype Mice

Next, investigators assessed whether exosomes isolated from the blood plasma of depressed patients also induce depressive behavior in mice and whether in vitro incubation of human exosomes with anti-ceramide antibodies clone S58-9 or ceramidase abrogates this effect.

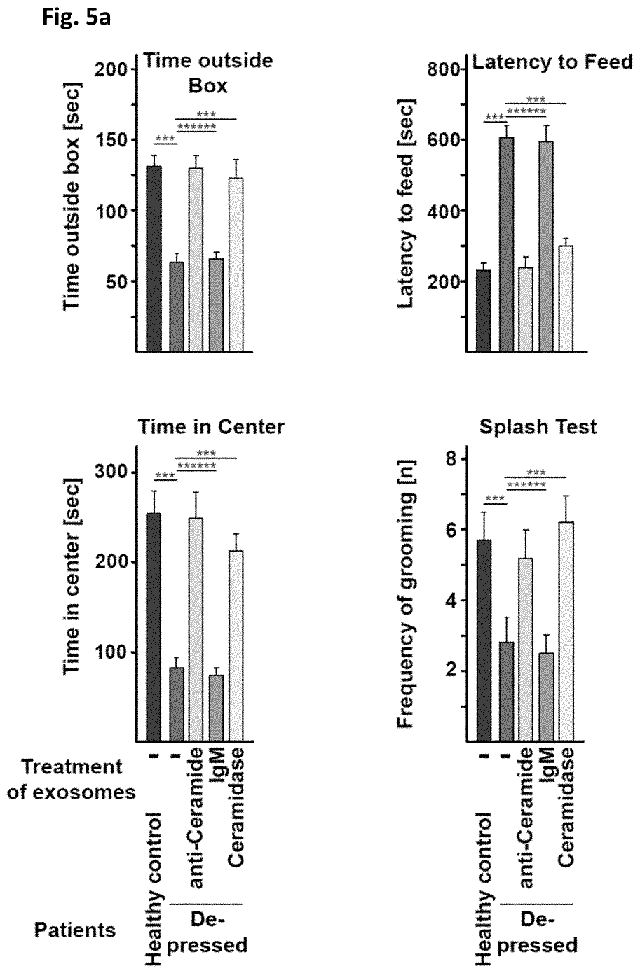

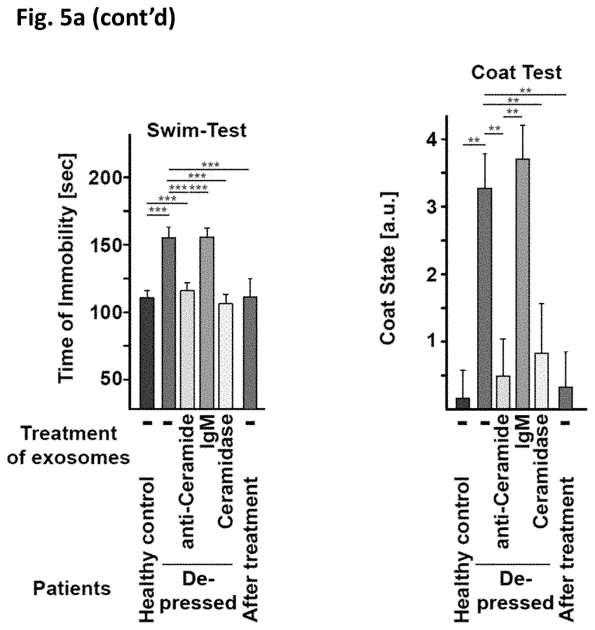

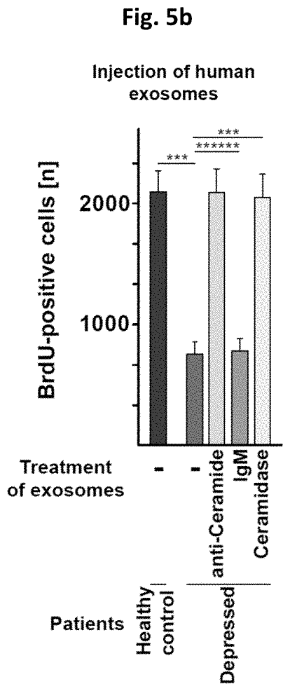

Exosomes were isolated from 150 μL blood plasma from patients with major depressive disorder (MDD), and from healthy control subjects. Exosomes from depressed patients were treated with anti-ceramide antibodies clone S58-9, control IgM, or recombinant ceramidase or were left untreated. Behavior (FIG. 5a) and neurogenesis (FIG. 5b) were determined to measure the occurrence of MDD in mice after injection of human exosomes from depressed patients. FIGS. 5a and b show the mean±SD from each 6 samples. *P<0.05, **P<0.01,***P<0.001, ANOVA.

The results show that exosomes from depressed patients, but not those from healthy individuals, induced depressed behavior and reduced neurogenesis as early as 24 h after injection (FIGS. 5a, 5b). Preincubation of the exosomes from the plasma of depressed patients with anti-ceramide antibodies clone S58-9 or ceramidase prevented their effects on mice, whereas control IgM exerted no effect (FIGS. 5a, 5b). Exosomes from healthy control subjects had no effects on neuronal proliferation and behavior (FIGS. 5a, 5b).

Embodiments can be described with reference to the following numbered clauses, with preferred features laid out in the dependent clauses.

1. A method of treating major depressive disorder (MDD) in a subject in need thereof, the method comprising administering to the subject an effective amount of an active agent that reduces an amount of ceramide in a peripheral compartment of the subject.

2. The method according to clause 1, wherein reducing the amount of ceramide in the peripheral compartment comprises degrading ceramide, neutralizing ceramide, and/or inhibiting synthesis of ceramide.

3. The method according to any preceding clause, wherein the agent is administered orally, intravenously, parenterally, intramuscularly, intraperitoneally, or transdermally.

4. The method according to any preceding clause, wherein the agent is administered orally or intravenously.

5. The method according to any preceding clause, wherein the subject is a mammal.

6. The method according to any preceding clause, wherein the subject is a human.

7. The method according to any preceding clause, wherein the active agent is selected from the group consisting of an antibody, a nucleic acid, a peptide, a protein, a small molecule compound, and combinations thereof.

8. The method according to any preceding clause, wherein the active agent is an anti-ceramide antibody.

9. The method according to any preceding clause, wherein the active agent is a humanized anti-ceramide antibody.

10. The method according to any of clauses 1-7, wherein the active agent is a ceramide-binding protein or a ceramide-binding peptide.

11. The method according to clause 10, wherein the ceramide-binding protein or the ceramide-binding peptide comprises a ceramide binding domain or a fragment, a derivative, or a variant thereof.

12. The method according to any of clause 1-7 or 10-11, wherein the ceramide binding domain is selected from the group consisting of an EF-hand calcium-binding motif, a heat shock chaperonin-binding motif (STI1), a sterol-binding domain (SCP2), and a tetratricopeptide repeat region motif.

13. The method according to any of clauses 1-7, wherein the active agent selectively degrades ceramide.

14. The method according to any of clauses 1-7 or 13, wherein the active agent is a neutral ceramidase, or a fragment, a derivative, or a variant thereof.

15. The method according to any of clauses 1-7 or 13-14, wherein the active agent is human neutral ceramidase.

16. The method according to any of clauses 1-7, wherein the active agent inhibits synthesis/release of ceramide.

17. The method according to any of clauses 1-7 or 16, wherein the active agent is a sphingomyelinase inhibitor.

18. The method according to any preceding clause, wherein the amount of ceramide in the peripheral compartment is reduced by at least 0.001 molar percentage relative to the amount of ceramide in the peripheral compartment prior to administering the active agent.

19. A method of reducing an amount of ceramide in neuronal tissue of a subject suffering from major depressive disorder (MDD), the method comprising peripherally administering to the subject an effective amount of an active agent that modulates ceramide.

20. The method according to claim 19, wherein modulating ceramide comprises degrading ceramide, neutralizing ceramide, and/or inhibiting synthesis of ceramide.

21. The method according to any of clauses 19-20, wherein peripheral administration comprises oral, intravenous, parenteral, intramuscular, intraperitoneal, and transdermal administration.

22. The method according to any of clauses 19-21, wherein the agent is administered orally or intravenously.

23. The method according to any of clauses 19-22, wherein the subject is a mammal.

24. The method according to any of clauses 19-23, wherein the subject is a human.

25. The method according to any of clauses 19-24, wherein the active agent that modulates ceramide is selected from the group consisting of an antibody, a nucleic acid, a peptide, a protein, a small molecule compound, and combinations thereof.

26. The method according to any of clauses 19-25, wherein the active agent is an anti-ceramide antibody.

27. The method according to any of clauses 19-26, wherein the agent is a humanized anti-ceramide antibody.

28. The method according to any of clauses 19-25, wherein the active agent is a ceramide-binding protein or a ceramide-binding peptide.

29. The method according to clause 28, wherein the ceramide-binding protein or the ceramide-binding peptide comprises a ceramide binding domain, or a fragment, a derivative, or a variant thereof.

30. The method according to any of clauses 28-29, wherein the ceramide binding domain is selected from the group consisting of an EF-hand calcium-binding motif, a heat shock chaperonin-binding motif (STI1), a sterol-binding domain (SCP2), and a tetratricopeptide repeat region motif.

31. The method according to any of clauses 19-25, wherein the active agent selectively degrades ceramide.

32. The method according to any of clauses 19-25 or 31, wherein the active agent is a neutral ceramidase, or a fragment, derivative, or variant thereof.

33. The method according to any of clauses 19-25 or 31-32, wherein the active agent is human neutral ceramidase.

34. The method according to any of clauses 19-25, wherein the active agent inhibits synthesis/release of ceramide.

35. The method according to any of clauses 19-25 or 34, wherein the active agent is a sphingomyelinase inhibitor.

36. The method according to any of clauses 19-35, wherein the amount of ceramide in the neuronal tissue is reduced by at least 0.001 molar percentage relative to the amount of ceramide in the neuronal tissue prior to administering the active agent.

37. The method according to any preceding clause, wherein the MDD is endogenous or reactive.

38. A method for identifying a candidate agent to treat major depressive disorder (MDD), the method comprising:

contacting ceramide with a test compound in a first sample;

contacting ceramide with a positive control in a second sample; and

measuring a concentration of functional ceramide in the first and second samples,

wherein a reduction in the concentration of functional ceramide in the first sample compared to the second sample indicates that test compound is a candidate agent for treating MDD.

39. The method of screening according to clause 38, wherein the positive controls are inhibitors of neutral sphingomyelinase or serine-palmitoyl-transferase.

40. The method according to any of clauses 38-39, wherein measuring the concentration of functional ceramide comprises measuring by kinase assay or mass spectrometry.

41. A method for identifying a candidate agent to treat major depressive disorder (MDD), the method comprising:

contacting an intact cell capable of synthesizing ceramide with a test compound in a first sample;

contacting an intact cell capable of synthesizing ceramide with a positive or negative control in a second sample; and

measuring a concentration of ceramide in the first and second samples, wherein a reduction in the concentration of ceramide in the first sample compared to the second sample indicates that the test compound is a candidate agent for treating MDD.

42. The method of screening according to clause 41, wherein the positive control is a ceramidase.

43. The method of screening according to any of clauses 41-42, wherein measuring the concentration of ceramide comprises measuring by kinase assay or mass spectrometry.

The forgoing description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

What is claimed is:

1. A method of treating or preventing major depressive disorder (MDD) or a symptom thereof in a subject suffering from MDD, exhibiting a symptom of MDD, or at risk for developing MDD, the method comprising administering to the subject an effective amount of an active agent that reduces an amount or functional activity of ceramide in a peripheral compartment of the subject.

2. The method according to claim 1, wherein reducing the amount of ceramide in the peripheral compartment comprises degrading ceramide, neutralizing ceramide, and/or inhibiting synthesis/release of ceramide.

3. The method according to claim 1, wherein the subject is a human.

4. The method according to claim 1, wherein the active agent is selected from the group consisting of an antibody, a nucleic acid, a peptide, a protein, a small molecule compound, and combinations thereof.

5. The method according to claim 4, wherein the active agent is an anti-ceramide antibody.

6. The method according to claim 4, wherein the active agent selectively degrades ceramide.

7. The method according to claim 4, wherein the active agent functions as a neutral ceramidase.

8. The method according to claim 4, wherein the active agent is a sphingomyelinase inhibitor.

9. The method according to claim 1, wherein the amount of ceramide in the peripheral compartment is reduced by at least 0.001 molar percentage relative to the amount of ceramide in the peripheral compartment prior to administering the active agent.

10. The method according to claim 1, wherein the MDD is endogenous or reactive.

11. A method for reducing an amount of ceramide in neuronal tissue of a subject suffering from major depressive disorder (MDD), the method comprising peripherally administering to the subject an effective amount of an active agent that modulates ceramide.

12. The method according to claim 11, wherein reducing the amount of ceramide in the peripheral compartment comprises degrading ceramide, neutralizing ceramide, and/or inhibiting synthesis/release of ceramide.

13. The method according to claim 11, wherein the subject is a human.

14. The method according to claim 11, wherein the active agent is selected from the group consisting of an antibody, a nucleic acid, a peptide, a protein, a small molecule compound, and combinations thereof.

15. The method according to claim 14, wherein the active agent is an anti-ceramide antibody.

16. The method according to claim 14, wherein the active agent selectively degrades ceramide.

17. The method according to claim 14, wherein the active agent functions as a neutral ceramidase.

18. The method according to claim 14, wherein the active agent is a sphingomyelinase inhibitor.

19. The method according to claim 11, wherein the amount of ceramide in the peripheral compartment is reduced by at least 0.001 molar percentage relative to the amount of ceramide in the peripheral compartment prior to administering the active agent.

20. A method for identifying a candidate agent to treat major depressive disorder (MDD), the method comprising:
contacting an intact cell capable of synthesizing/releasing ceramide with a test compound in a first sample;
optionally contacting an intact cell capable of synthesizing/releasing ceramide with a positive or negative control in a second sample; and
measuring a concentration of ceramide in the first and optionally second samples, wherein a reduction in the concentration of ceramide in the first sample compared to the second sample or control indicates that the test compound is a candidate agent for treating MDD.


SEQUENCE LISTING (30 sequences)

SEQ ID NO: 1 - protein, 10 aa, Artificial Sequence
  Anti-ceramide antibody heavy chain variable region CDR1
GYTFTDHTIH

SEQ ID NO: 2 - protein, 17 aa, Artificial Sequence
  Anti-ceramide antibody heavy chain variable region CDR2
YNYPRDGSTKYNEKFKG

SEQ ID NO: 3 - protein, 11 aa, Artificial Sequence
  Anti-ceramide antibody heavy chain variable region CDR3
GFITTVVPSAY

SEQ ID NO: 4 - protein, 11 aa, Artificial Sequence
  Anti-ceramide antibody light chain variable region CDR1
RASKSISKYLA

SEQ ID NO: 5 - protein, 7 aa, Artificial Sequence
  Anti-ceramide antibody light chain variable region CDR2
SGSTLQS

SEQ ID NO: 6 - protein, 9 aa, Artificial Sequence
  Anti-ceramide antibody light chain variable region CDR3
QQHNEYPWT

SEQ ID NO: 7 - protein, 10 aa, Artificial Sequence
  Anti-ceramide antibody heavy chain variable region CDR1
GYAFSSYWMN

SEQ ID NO: 8 - protein, 17 aa, Artificial Sequence
  Anti-ceramide antibody heavy chain variable region CDR2
QIYPGDGDTNYNGKFKG

SEQ ID NO: 9 - protein, 10 aa, Artificial Sequence
  Anti-ceramide antibody heavy chain variable region CDR3
RCYYGLYFDV

SEQ ID NO: 10 - protein, 11 aa, Artificial Sequence
  Anti-ceramide antibody light chain variable region CDR1
KASQDINRYLS

SEQ ID NO: 11 - protein, 7 aa, Artificial Sequence
  Anti-ceramide antibody light chain variable region CDR2
RANRLVD

SEQ ID NO: 12 - protein, 9 aa, Artificial Sequence
  Anti-ceramide antibody light chain variable region CDR3
LQYDEFPYT

SEQ ID NO: 13 - protein, 10 aa, Artificial Sequence
  Anti-ceramide antibody heavy chain variable region CDR1
GYTFTSYWMH

SEQ ID NO: 14 - protein, 16 aa, Artificial Sequence
  Anti-ceramide antibody heavy chain variable region CDR2
YINPSSGYTKYNQFKD

SEQ ID NO: 15 - protein, 8 aa, Artificial Sequence
  Anti-ceramide antibody heavy chain variable region CDR3
GGYYGFAY

SEQ ID NO: 16 - protein, 10 aa, Artificial Sequence
  Anti-ceramide antibody light chain variable region CDR1
SASSSVSYMY

SEQ ID NO: 17 - protein, 7 aa, Artificial Sequence
  Anti-ceramide antibody light chain variable region CDR2
LTSNLAS

SEQ ID NO: 18 - protein, 9 aa, Artificial Sequence
  Anti-ceramide antibody light chain variable region CDR3
QQWSSNPLT

SEQ ID NO: 19 - protein, 10 aa, Artificial Sequence
  Anti-ceramide antibody heavy chain variable region CDR1
GFSLTGYGVH

SEQ ID NO: 20 - protein, 16 aa, Artificial Sequence
  Anti-ceramide antibody heavy chain variable region CDR2
VIWSGGSTDYNAAFIS

SEQ ID NO: 21 - protein, 10 aa, Artificial Sequence
  Anti-ceramide antibody heavy chain variable region CDR3
NYGYDYAMDY

SEQ ID NO: 22 - protein, 11 aa, Artificial Sequence
  Anti-ceramide antibody light chain variable region CDR1
RASQSIGTSIH

SEQ ID NO: 23 - protein, 7 aa, Artificial Sequence
  Anti-ceramide antibody light chain variable region CDR2
YASESIS

SEQ ID NO: 24 - protein, 9 aa, Artificial Sequence
  Anti-ceramide antibody light chain variable region CDR3
QQSNSWPFT

SEQ ID NO: 25 - protein, 10 aa, Artificial Sequence
  Anti-ceramide antibody heavy chain variable region CDR1
GYTFTNYWMH

SEQ ID NO: 26 - protein, 17 aa, Artificial Sequence
  Anti-ceramide antibody heavy chain variable region CDR2
AIYPGDSDTSYNQKFKG

SEQ ID NO: 27 - protein, 7 aa, Artificial Sequence
  Anti-ceramide antibody heavy chain variable region CDR3
GLYYGYD

SEQ ID NO: 28 - protein, 16 aa, Artificial Sequence
  Anti-ceramide antibody light chain variable region CDR1
KSSQSLIDSDGKTFLN

SEQ ID NO: 29 - protein, 7 aa, Artificial Sequence
  Anti-ceramide antibody light chain variable region CDR2
LVSKLDS

SEQ ID NO: 30 - protein, 9 aa, Artificial Sequence
  Anti-ceramide antibody light chain variable region CDR3
WQGTHFPYT